(19) United States
(12) Patent Application Publication
(10) Pub. No.: US 2024/0197911 A1
(43) Pub. Date: Jun. 20, 2024

(54) Oral Pharmaceutical Composition, Method for Fabricating Thereof, and Kit for Treating Cancer

(71) Applicant: National Tsing Hua University, Hsinchu (TW)

(72) Inventors: Hsing-Wen Sung, Hsinchu (TW); Kuan-Hung Chen, Hsinchu (TW); Tun-Yu Huang, Hsinchu (TW); Nhien Hoang To Nguyen, Hsinchu (TW); Yu-Jung Lin, Taipei (TW); Yu-Tzu Yu, Hsinchu (TW)

(21) Appl. No.: 18/124,129

(22) Filed: Mar. 21, 2023

(30) Foreign Application Priority Data
Dec. 8, 2022 (TW) 111147250

(51) Int. Cl. A61K 47/69; A61K 9/00; A61K 31/12; A61K 31/203; A61K 31/352; A61K 31/44; A61K 31/704; A61K 39/395; A61P 35/00

(52) U.S. Cl. CPC A61K 47/6949; A61K 9/0053; A61K 31/12; A61K 31/203; A61K 31/352; A61K 31/44; A61K 31/704; A61K 39/3955; A61P 35/00

(57) ABSTRACT

An oral pharmaceutical composition includes a metal complex and an outer layer. The metal complex is formed by coordinate bonding of an organic compound to a metal ion, and the organic compound is an aromatic compound and includes at least two oxygen atoms. The outer layer includes a β-glucan, and the β-glucan is dispersed outside the metal complex and coordinately bonded to the metal complex to form the outer layer.

### CROSS-REFERENCE TO RELATED APPLICATIONS

This application claims priority to Taiwanese Patent Application No. 111147250, filed Dec. 8, 2022, the disclosure of which is hereby incorporated by reference in its entirety.

### BACKGROUND OF THE INVENTION

### Field of the Invention

The present disclosure relates to a composition, a method for fabricating thereof and a kit including the composition. More particularly, the present disclosure relates to a pharmaceutical composition with a special physical form, a method for fabricating thereof and a kit for treating cancer.

### Description of Related Art

Drugs are substances that have therapeutic effects for curing diseases, reducing suffering of patients, or preventing human diseases. Drugs include natural ingredients, chemically synthesized substances, and biological agents. The general modes of administration include injection administrations (such as intravenous injection, intramuscular injection or subcutaneous injection, etc.), oral administrations (such as oral administration through the gastrointestinal tract, sublingual tablets and oral tablets, etc.) and external administrations (such as transdermal mucosal medication, transdermal absorption medication, transnasal mucosa or pulmonary respiratory tract medication, etc.).

Different modes of administration have their own advantages and disadvantages. Oral administration is to swallow the drug through the gastrointestinal mucosa and transport it to various parts of the body through the bloodstream to make it function in the body. Oral administration eliminates the need for needles and is convenient to use, which is conducive to patient self-management. In addition, the cost of oral drug production is low, and the price of oral drug is relatively cheap. Therefore, the oral administration is considered a promising way of administration.

However, there are still problems with oral administration, such as slow and irregular drug absorption. Oral administration is also prone to encounter the mucosal barrier formed by tightly arranged epithelial cells in the intestine, reducing its effectiveness. Therefore, how to develop a new type of oral pharmaceutical composition that can effectively deliver the loaded active drug to the target in the body, so as to improve the drug effect, has become important development goals in the field of pharmacy today.

### SUMMARY OF THE INVENTION

According to one aspect of the present disclosure, an oral pharmaceutical composition includes a metal complex and an outer layer. The metal complex is formed by coordinate bonding of an organic compound to a metal ion, and the organic compound is an aromatic compound and includes at least two oxygen atoms. The outer layer includes a β-glucan, and the β-glucan is dispersed outside the metal complex and coordinately bonded to the metal complex to form the outer layer.

According to another aspect of the present disclosure, a method for fabricating an oral pharmaceutical composition includes steps as follows. A mixed solution is provided, wherein the mixed solution includes an organic compound, a metal ion and a β-glucan, and the organic compound is an aromatic compound and includes at least two oxygen atoms. A synthesis step is performed, wherein energy is provided to the mixed solution, then the organic compound coordinates with the metal ion to form a metal complex, and the β-glucan coordinates with the metal complex to form the oral pharmaceutical composition.

According to still another aspect of the present disclosure, a kit for treating cancer is provided. The kit for treating cancer includes the oral pharmaceutical composition according to the aforementioned aspect and a pharmaceutically acceptable carrier.

### BRIEF DESCRIPTION OF THE DRAWINGS

The present disclosure can be more fully understood by reading the following detailed description of the embodiment, with reference made to the accompanying drawings as follows:

FIG. 1 is a structural schematic view showing an oral pharmaceutical composition according to the present disclosure.

FIG. 2 is a flow chart showing a method for fabricating an oral pharmaceutical composition according to the present disclosure.

FIG. 3A, FIG. 3B, FIG. 3C, FIG. 3D, FIG. 3E, FIG. 3F, FIG. 3G, FIG. 3H, FIG. 3I and FIG. 3J show analysis results of the structure and characteristics of Comparative Example and Example 1 of the oral pharmaceutical composition of the present disclosure.

FIG. 4A, FIG. 4B, FIG. 4C, FIG. 4D, FIG. 4E, FIG. 4F, FIG. 4G, FIG. 4H, FIG. 4I, FIG. 4J, FIG. 4K and FIG. 4L show analysis results of the cell regulation process of Comparative Example and Example 1 of the oral pharmaceutical composition of the present disclosure in RAW 264.7 cells.

FIG. 5A, FIG. 5B, FIG. 5C and FIG. 5D show analysis results of the biodistribution of Comparative Example and Example 1 of the oral pharmaceutical composition of the present disclosure in orthotopic pancreatic ductal adenocarcinoma (PDAC) mice.

FIG. 6A and FIG. 6B show analysis results of the absorption mechanism of Example 1 of the oral pharmaceutical composition of the present disclosure.

FIG. 7A, FIG. 7B, FIG. 7C, FIG. 7D, FIG. 7E, FIG. 7F, FIG. 7G, FIG. 7H, FIG. 7I and FIG. 7J show analysis results of the antitumor efficacy and tumor microenvironment regulation of Example 1 of the oral pharmaceutical composition of the present disclosure in the orthotopic PDAC model.

FIG. 8A, FIG. 8B, FIG. 8C, FIG. 8D, FIG. 8E, FIG. 8F, FIG. 8G, FIG. 8H, FIG. 8I, FIG. 8J and FIG. 8K show analysis results of the antitumor efficacy and tumor microenvironment regulation of Example 2 of the kit for treating cancer of the present disclosure in the orthotopic PDAC model.

FIG. 9A, FIG. 9B, FIG. 9C, FIG. 9D, FIG. 9E, FIG. 9F, FIG. 9G, FIG. 9H and FIG. 9I show analysis results of the antitumor efficacy and tumor microenvironment regulation of Example 1 of the oral pharmaceutical composition of the present disclosure and Example 2 of the kit for treating cancer of the present disclosure in the advanced PDAC model.

FIG. 10A is a schematic view showing the oral pharmaceutical composition and the kit for treating cancer of the present disclosure used in the treatment of PDAC model.

FIG. 10B is a schematic view showing a mechanism of intestinal absorption of the oral pharmaceutical composition of the present disclosure mediated by M cells.

FIG. 10C is a schematic view showing a mechanism of the oral pharmaceutical composition and the kit for treating cancer of the present disclosure targeting primary tumor and metastatic tumor.

FIG. 10D is a schematic view showing a mechanism of lysosomal translocation and lysosomal exocytosis of the oral pharmaceutical composition of the present disclosure.

FIG. 10E is a schematic view showing an action mechanism of the oral pharmaceutical composition and the kit for treating cancer of the present disclosure remodeling the tumor microenvironment.

### DESCRIPTION OF THE INVENTION

The following descriptions of particular embodiments and examples are provided by way of illustration and not by way of limitation. Those skilled in the art will readily recognize a variety of noncritical parameters that could be changed or modified to yield essentially similar results.

Unless otherwise stated, the meanings of the scientific and technical terms used in the specification are the same as those of ordinary skill in the art. Furthermore, the nouns used in this specification are intended to cover the singular and plural terms of the term unless otherwise specified.

The term “about” means that the actual value falls within the acceptable standard error of the average, as determined by person having ordinary skill in the art. The scope, number, numerical values, and percentages used herein are modified by the term “about” unless example or otherwise stated. Therefore, unless otherwise indicated, the numerical values or parameters disclosed in the specification and the claims are approximate values and can be adjusted according to requirements.

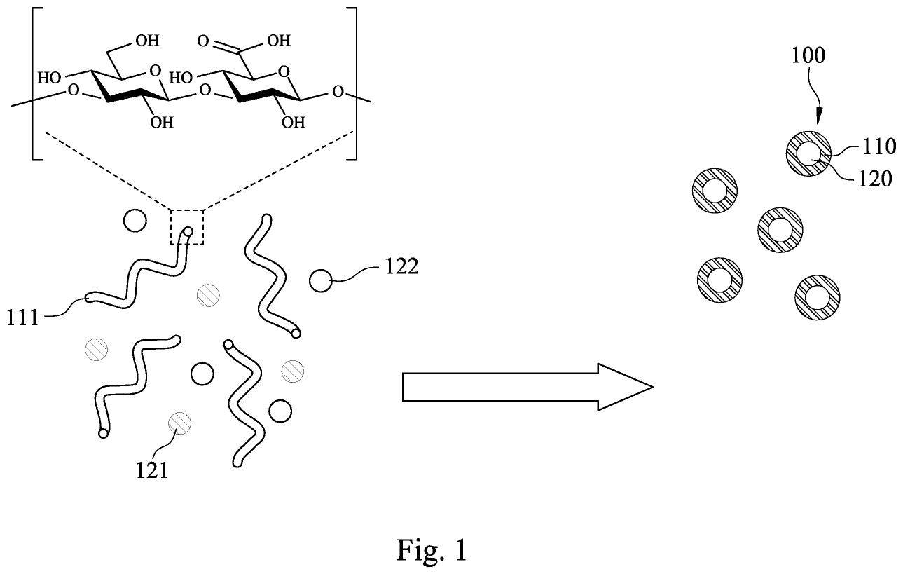

Reference is made to FIG. 1, which is a structural schematic view showing an oral pharmaceutical composition 100 according to the present disclosure. The oral pharmaceutical composition 100 includes a metal complex 120 and an outer layer 110. The metal complex 120 is formed by coordinate bonding of an organic compound 122 to a metal ion 121, and the organic compound 122 is an aromatic compound and includes at least two oxygen atoms. In greater detail, the metal complex 120 is formed by the metal ion 121 with empty orbital bonding with the organic compound 122 that provides lone pair electrons by coordination bond. The outer layer 110 includes a β-glucan 111, and the β-glucan 111 is dispersed outside the metal complex 120 and coordinately bonded to the metal complex 120 to form the outer layer 110.

The oral pharmaceutical composition 100 can be a sphere, and the average particle size of the sphere is greater than or equal to 80 nm and less than 1 μm. The metal ion 121 can be zinc ion (Zn²⁺), manganese ion (Mn²⁺), cobalt ion (Co²⁺), iron ion (Fe³⁺) or nickel ion (Ni²⁺). The organic compound 122 can be doxorubicin (DOX), retinoic acid, curcumin, quercetin or sorafenib, and the structural formula of the organic compound 122 is shown in Table 1 below.

TABLE 1

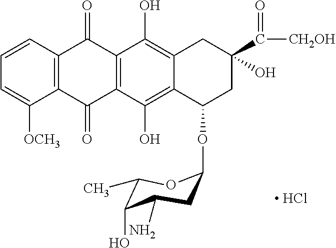

doxorubicin

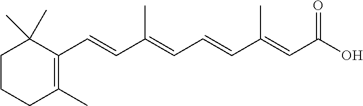 [SMILES: CC1=C(/C=C/C(C)=C/C=C/C(C)=C/C(=O)O)C(C)(C)CCC1]

retinoic acid

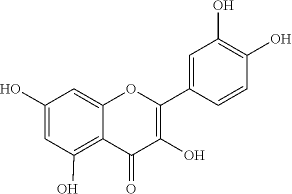 [SMILES: O=c1c(O)c(-c2ccc(O)c(O)c2)oc2cc(O)cc(O)c12]

quercetin

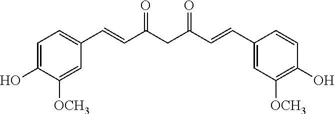 [SMILES: COc1cc(/C=C/C(=O)CC(=O)/C=C/c2ccc(O)c(OC)c2)ccc1O]

curcumin

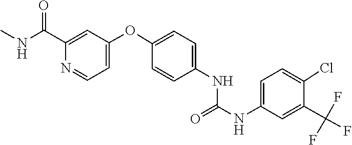 [SMILES: CNC(=O)c1cc(Oc2ccc(NC(=O)Nc3ccc(Cl)c(C(F)(F)F)c3)cc2)ccn1]

sorafenib

Therefore, the oral pharmaceutical composition 100 of the present disclosure forms the metal complex 120 stably by coordination bond between the metal ion 121 and the organic compound 122, so that the organic compound 122 can remain stable in the extreme intestinal environment. The outer layer 110 of the oral pharmaceutical composition 100 can target the microfold cells (M cells) of the intestinal tract, so that the oral pharmaceutical composition 100 can effectively overcome the epithelial barrier of the small intestine, increase oral absorption efficiency, be absorbed into the body through the lymphatic system, and then undergo subsequent phagocytosis by endogenous macrophages that reside in the intestinal lymphatic system (ILS). The macrophages respond to chemokines/cytokines released from the lesion, and then home in the oral pharmaceutical composition 100 carried on the lesion site and release the organic compound 122.

Accordingly, when the organic compound 122 in the oral pharmaceutical composition 100 is an anticancer drug, the oral pharmaceutical composition 100 can be subsequently used as a kit for treating cancer (not shown). The kit for treating cancer of the present disclosure includes the aforementioned oral pharmaceutical composition 100 and a pharmaceutically acceptable carrier. Specifically, the organic compound 122 is the anticancer drug, such as doxorubicin, retinoic acid, curcumin, quercetin or sorafenib. The kit for treating cancer can further include an immune checkpoint inhibitor. The immune checkpoint inhibitor can be selected from the group consisting of PD-L1 antibody, PD-1 antibody, CTLA-4 antibody and TIM-3 antibody.

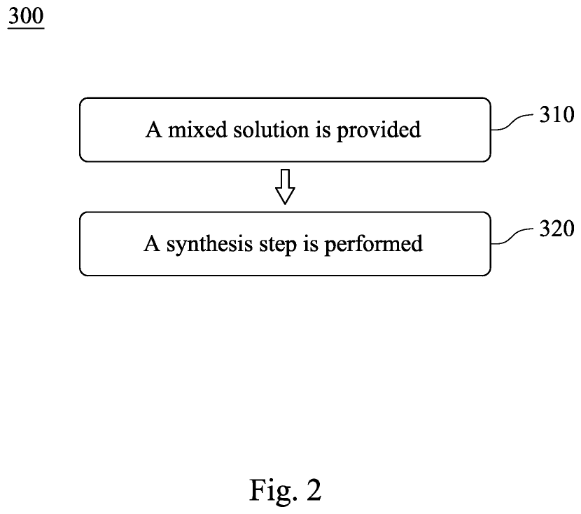

Reference is made to FIG. 2, which is a flow chart showing a method for fabricating an oral pharmaceutical composition 300 according to the present disclosure. The method for fabricating the oral pharmaceutical composition 300 includes Step 310 and Step 320.

In Step 310, a mixed solution is provided. The mixed solution includes an organic compound, a metal ion and a β-glucan, and the organic compound is an aromatic compound and includes at least two oxygen atoms. The molar ratio of the organic compound and the metal ion in the mixed solution can be 0.5:1 to 8:1. The organic compound can be doxorubicin (DOX), retinoic acid, curcumin, quercetin or sorafenib, and the structural formula of the organic compound is shown in Table 1. The metal ion is formed by a dissociation of a metal salt in water, and the metal ion can be zinc ion (Zn²⁺), manganese ion (Mn²⁺), cobalt ion (Co²⁺), iron ion (Fe³⁺) or nickel ion (Ni²⁺).

In Step 320, a synthesis step is performed. Energy is provided to the mixed solution, then the organic compound coordinates with the metal ion to form a metal complex, and the β-glucan coordinates with the metal complex to form the oral pharmaceutical composition. Specifically, energy can be provided by a microwave or a heating.

Therefore, the method for fabricating the oral pharmaceutical composition 300 of the present disclosure is a simple one-pot method for fabricating the oral pharmaceutical composition. By mixing the organic compound, the metal salt and the β-glucan to form the mixed solution, and then providing energy to the mixed solution, so that the organic compound coordinates with the metal ion to form the metal complex, and the β-glucan coordinates with the metal complex to form the oral pharmaceutical composition with the outer layer including the β-glucan which is dispersed outside the metal complex.

In the following, reference will now be made in detail to the present embodiments of the present disclosure, experiments and examples of which are illustrated in the accompanying drawings. The effect and the mechanism of the oral pharmaceutical composition of the present disclosure are demonstrated by Examples and Comparative Example. However, the present disclosure is not limited thereto.

### EXAMPLES

### I. The Oral Pharmaceutical Composition of the Present Disclosure and the Method for Fabricating Thereof

### 1.1. Fabrication of the Oral Pharmaceutical Composition

Example 1 of the oral pharmaceutical composition is first prepared, wherein the organic compound used is doxorubicin (hereinafter referred to as “DOX”), and the metal ion is zinc ion (Zn²⁺). The β-glucan used has a molecular weight of about 50 kDa and has a negative charge (−16.6+1.3 mV), which can be extracted from yeast, especially Saccharomyces cerevisiae. Further, the yeast can be destroyed by acid and alkali, and its cytoplasm can be removed by isopropyl alcohol and acetone solution to obtain the β-glucan cell-wall shell. Then the β-glucan is obtained by degradation of the β-glucan cell-wall shell.

0.01 mmol of zinc acetate dihydrate, 0.01 mmol of DOX hydrochloride and 0.1 w/v % β-glucan are dissolved in 10 mL of dimethylformamide (DMF) at room temperature to obtain the mixed solution. The mixed solution is transferred to a microwave glass vial, which is then placed in a microwave system (Monowave 400, Anton Paar) and heated to 160° C. for 1 hour to perform the synthesis step. After the synthesis step is completed, the mixed solution is placed in a rotary evaporator (N-1200A, EYELA) for evaporating the DMF solvent therein under a gradually decreasing pressure at 60° C. to obtain Example 1 of the oral pharmaceutical composition (hereinafter referred to as “Example 1”). Example 1 is rinsed twice in ethanol and once in deionized water, and collected by centrifugation at 18,000 rpm for 10 minutes. Comparative Example is also prepared using a similar procedure of Example 1 but in the absence of β-glucan.

### 1.2. Analysis of Structure and Characteristics of the Oral Pharmaceutical Composition

The UV-vis absorbance spectra of Example 1, Comparative Example and free DOX in DMF are obtained using a SpectraMax M5 Microplate Reader (Molecular Devices). The chemical structures of Example 1 and Comparative Example are analyzed by Fourier-transform infrared spectroscopy (FT-IR) (Nicolet™ iS™50 spectrometer, Thermo Fisher Scientific).

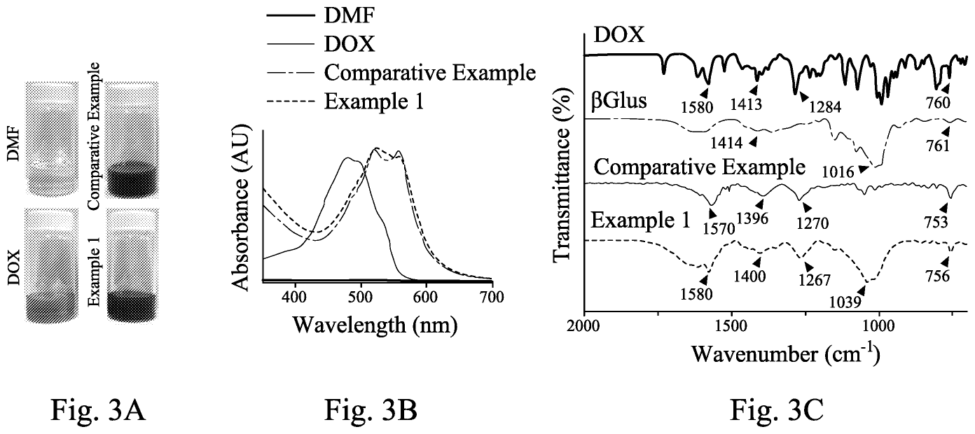

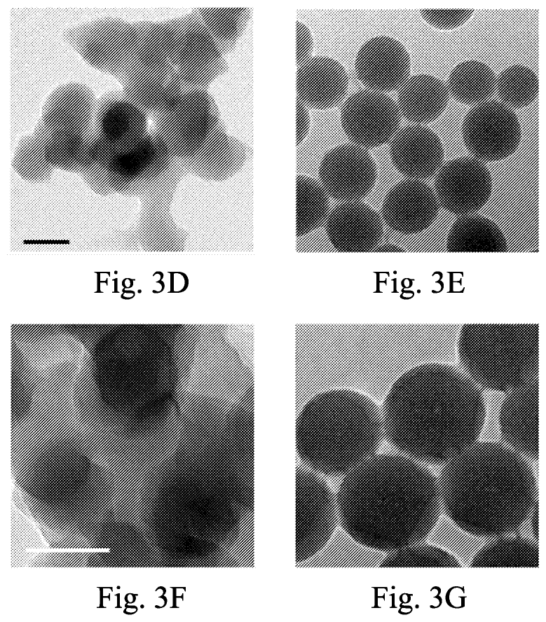

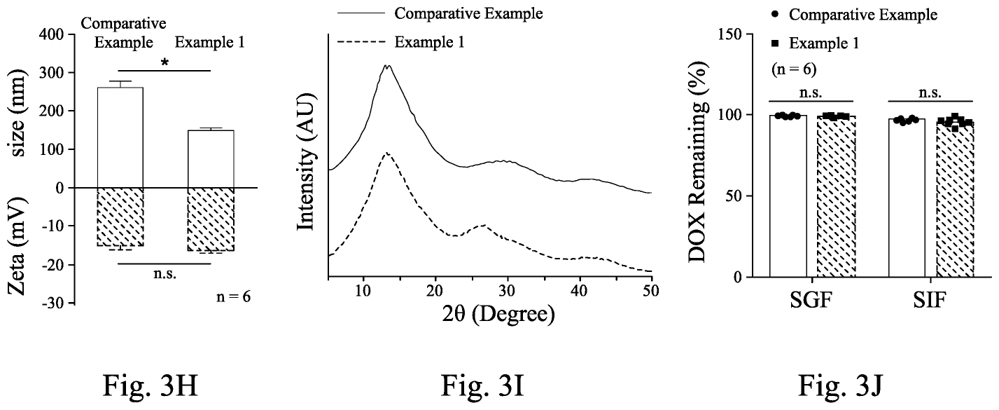

Reference is made to FIG. 3A and FIG. 3B, which are respectively photographs and UV-vis absorbance spectra of free DOX, Comparative Example and Example 1 dissolved in DMF, wherein DOX represents free DOX. In FIG. 3A, the solution including free DOX is red, while the solution including Comparative Example or Example 1 is purple. In FIG. 3B, the solution including free DOX has a characteristic peak at 480 nm, while the characteristic peak is redshifted to 534 nm in the solution including Comparative Example or Example 1. Compared with the solution including free DOX, the observed redshift in wavelength and change in color in Comparative Example and Example 1 suggest the formation of coordination bond between Zn²⁺ and DOX. Comparative Example is formed by the complexation of Zn²⁺ and DOX, and Example 1 is formed by the complexation of Zn²⁺, DOX and β-glucan.

Reference is made to FIG. 3C, which shows FT-IR spectra of free DOX, β-glucan, Comparative Example and Example 1, wherein DOX represents free DOX, and βGlus represents β-glucan. In FIG. 3C, the FT-IR spectrum of Comparative Example includes characteristic peaks at almost the same wavenumbers as free DOX, while the FT-IR spectrum of Example 1 includes characteristic peaks of both free DOX and β-glucan. The formation of Comparative Example and Example 1 can be further confirmed by FT-IR spectra.

In addition, the formulation of Example 1 is optimized with a view to maximizing the amount of the encapsulated organic compound (DOX), by controlling the feeding molar ratio of Zn²⁺ to DOX under the condition of fixing the total amount of β-glucan to prepare the Example 1 of the present disclosure. To determine loading content (LC) and loading efficiency (LE) of the organic compound (DOX) in Example 1, weighted samples are dissolved in dimethyl sulfoxide (DMSO) to release the encapsulated drug (DOX), whose concentration is measured using SpectraMax M5 Microplate Reader. The following equations are used to calculate the LC and the LE of DOX in Example 1.

$$LC ( \% ) = \frac{weight of DOX in Example 1}{weight of Example 1} \times 100 \% ; and \qquad equation I$$

$$LE ( \% ) = \frac{total amount of DOX added - free DOX}{total amount of DOX added} \times 100 \% . \qquad equation II$$

Please refer to Table 2, which shows the LC and the LE of the Example 1 of the oral pharmaceutical composition that are synthesized using various molar ratios of Zn²⁺ (metal ion) to DOX (organic compound).

TABLE 2

| Molar ratio of Zn²⁺ to DOX | LC (%) | LE (%) |
|---|---|---|
| 1.0:0.25 | N/A | N/A |
| 1.0:0.5 | 7.2 ± 0.7 | 81.2 ± 4.5 |
| 1.0:1.0 | 20.7 ± 3.6 | 74.8 ± 5.2 |
| 1.0:2.0 | 21.1 ± 2.8 | 55.0 ± 0.7 |

In Table 2, when the molar ratio of Zn²⁺ and DOX is 1.0:0.25, no nanoparticles are formed. When the molar ratio of Zn²⁺ and DOX is 1.0:0.5 to 1.0:2.0 (even up to 1.0:8.0), Example 1 can be formed. As the DOX feeding molar ratio increases, the LC of the as-formed Example 1 increases, reaching a maximum at a Zn²⁺ to DOX molar ratio of 1.0:1.0, which correspond to a DOX LC of 20.7±3.6% and a DOX LE of 74.8±5.2%. This formulation is therefore used to prepare Example 1 and Comparative Example for use in subsequent studies.

Morphologies of Example 1 and Comparative Example are observed under a transmission electron microscope (TEM) (JEM-2100F, JEOL Technics). The particle sizes and zeta potentials of Comparative Example and Example 1 in DI water are measured using DLS (Zetasizer, 3000 HS, Malvern Instruments), and crystalline structures of Comparative Example and Example 1 are determined using an X-ray diffractometer (D8A25, Bruker).

Reference is made to FIG. 3D to FIG. 3I. FIG. 3D and FIG. 3F are the TEM images of Comparative Example, FIG. 3E and FIG. 3G are the TEM images of Example 1, FIG. 3H shows analysis results of particle sizes and zeta potentials of Comparative Example and Example 1, and FIG. 3I shows analysis results of X-ray powder diffraction (XRD) patterns of Comparative Example and Example 1, wherein n.s. represents no significant difference (P>0.05), * represents P<0.05.

The TEM images in FIG. 3D to FIG. 3G show that Comparative Example tends to aggregate together in terms of morphology and structure, while monodispersed spherical Example 1 can be observed. In FIG. 3G, a bright layer of a substance that is likely to be β-glucan owing to their low electron density is observed on the surface of each Example 1, revealing that β-glucan is dispersed on the surface of Example 1 to form the outer layer.

In FIG. 3H, the average particle size of the monodispersed Example 1 is significantly smaller than the aggregated Comparative Example (P<0.05). In FIG. 3I, no sharp peak is observed in the XRD patterns of either Comparative Example or Example 1, implying that their crystal structures are amorphous.

To evaluate the stability of Example 1 under gastrointestinal (GI) conditions, Comparative Example and Example 1 are individually incubated in simulated gastric fluid (SGF) and simulated intestinal fluid (SIF) at 37° C. The SGF is an HCl solution at pH 2.0 that contains 0.2% NaCl (by w/v %) and 0.5 mg/mL pepsin, and the SIF is a solution at pH 7.0 that contains 5 mg/mL bile extract and 1.6 mg/mL lipase. At pre-determined times, samples are collected and centrifuged at 18,000 rpm for 10 minutes, and the concentration of DOX that is released into the supernatant is analyzed using a SpectraMax M5 Microplate Reader.

Reference is made to FIG. 3J, which shows analysis results of the stability of Comparative Example and Example 1 in the simulated GI environment. The results show that no significant leakage of DOX from neither Comparative Example nor Example 1 is detected in SGF or SIF, suggesting stability of Example 1 in the simulated GI environment and showing the potential of Example 1 to remain intact in GI in vivo.

### 1.3. Cell Regulation Process of the Oral Pharmaceutical Composition in Macrophages

The regulation and stability of macrophage (hereinafter referred to as “Mφ”) in the body is very important. Too many and too few Mφ may cause many immune-related diseases. Mφ responds to cytokines (CSF-1, VEGF, PDGF, TNF, etc.) released by tumor cells, gathers around the tumor, and even transpasses deep into the tumor. As a class of phagocytic cells, Mφ can take up drug-encapsulated nanoparticles, functioning as Mφ-hitchhiked drug delivery vehicles. Most of the current studies using Mφ as a drug carrier are co-cultured with the drug carrier in vitro, and then injected back into the body by intravenous injection. Although research supports the efficacy of this therapy, it is limited by concerns about its complexity, time-consuming, and high risk of contamination.

To investigate whether the oral pharmaceutical composition of the present disclosure can be phagocytosed and carried by Mφ, the cellular uptake and trafficking of free DOX, Comparative Example or Example 1 by Mφ in vitro are observed first. RAW264.7 cells, a murine Mφ cell line, are seeded in an eight-well chamber slide (ibidi) at a density of 5×10⁴ cells per well and incubated overnight. The cells are then treated with each of the test samples at an equivalent dose of DOX (10 μg/mL). Following incubation for pre-determined periods (0, 2, 4, and 6 hours), test cells are washed with Dulbecco's phosphate-buffered saline (DPBS) and stained with LysoTracker™ Deep Red (Invitrogen) and Hoechst 33342 (Abcam) for 30 minutes in the incubator. The stained cells are then examined using confocal laser scanning microscopy (CLSM) (LSM 780, Carl Zeiss) to track the localization and accumulation of free DOX, the DOX fluorescent signal of Comparative Example or Example 1, and to determine the cell regulation process of free DOX, Comparative example and Example 1 in the RAW264.7 cells.

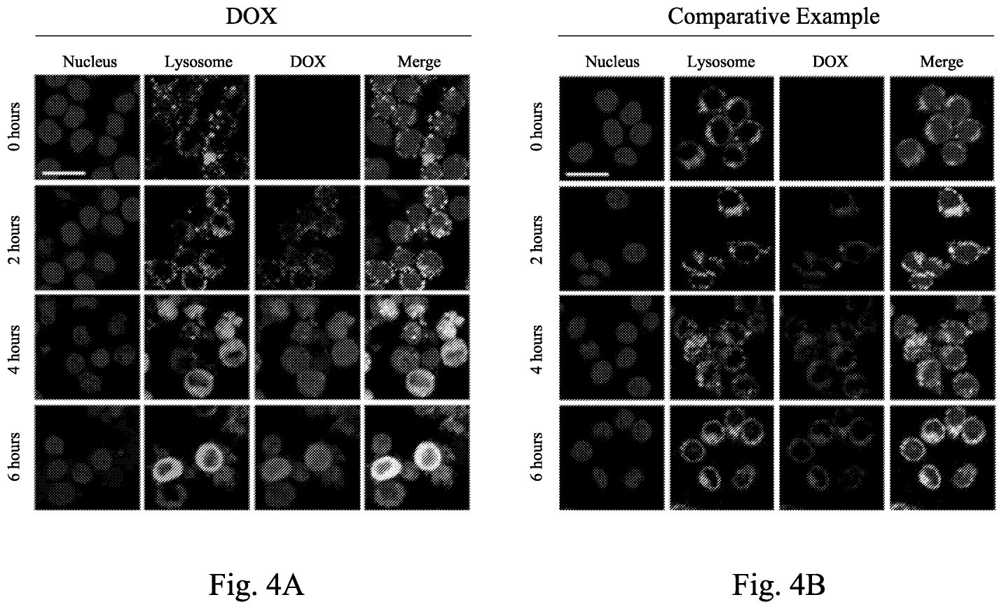

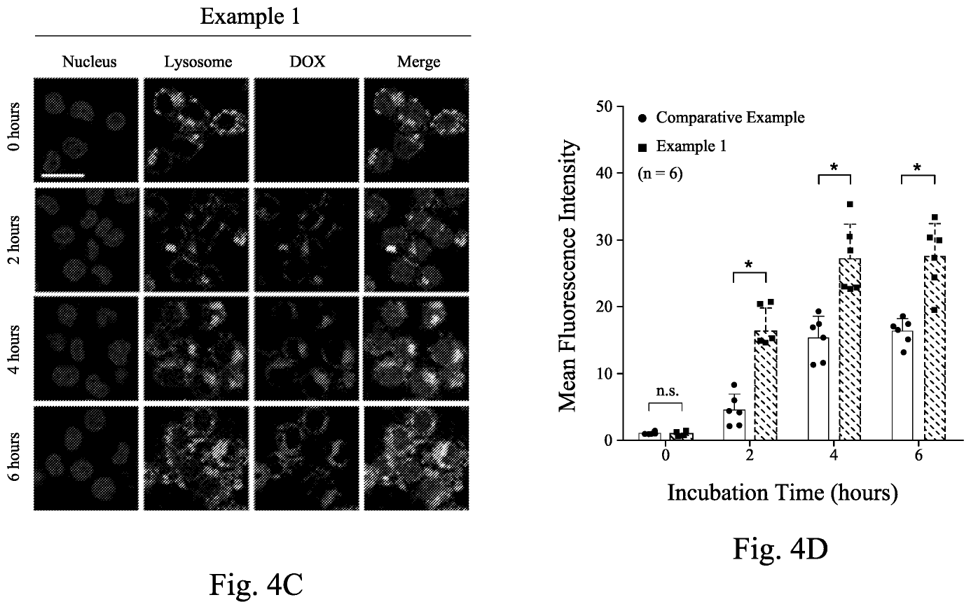

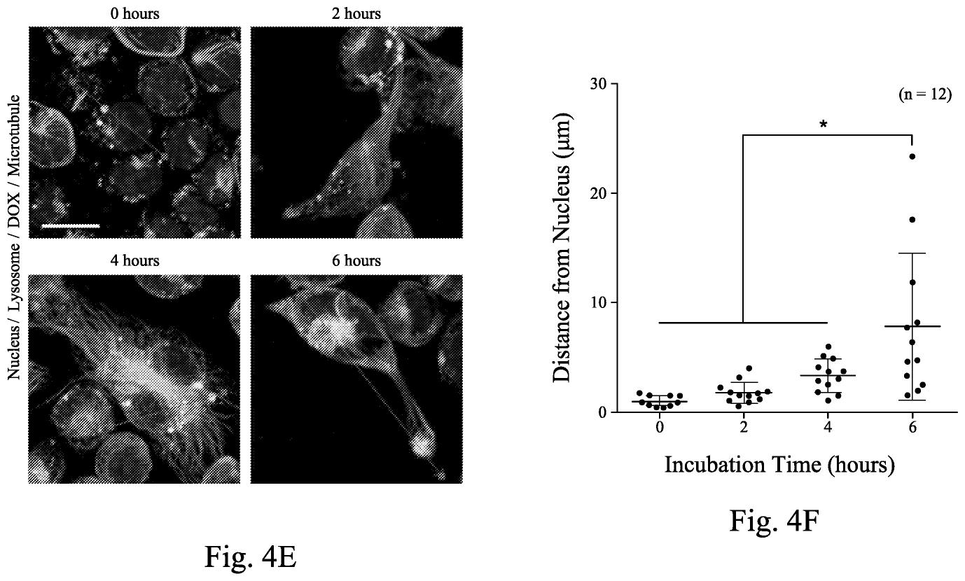

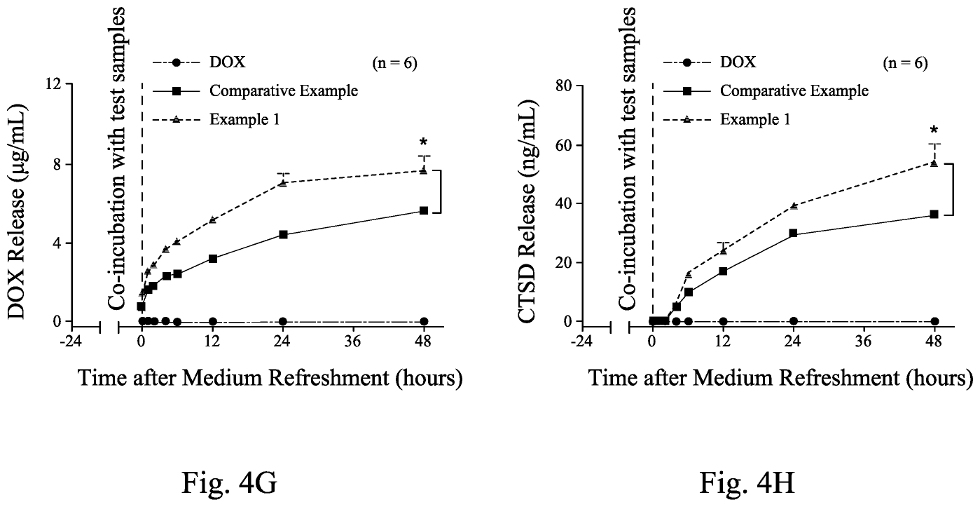

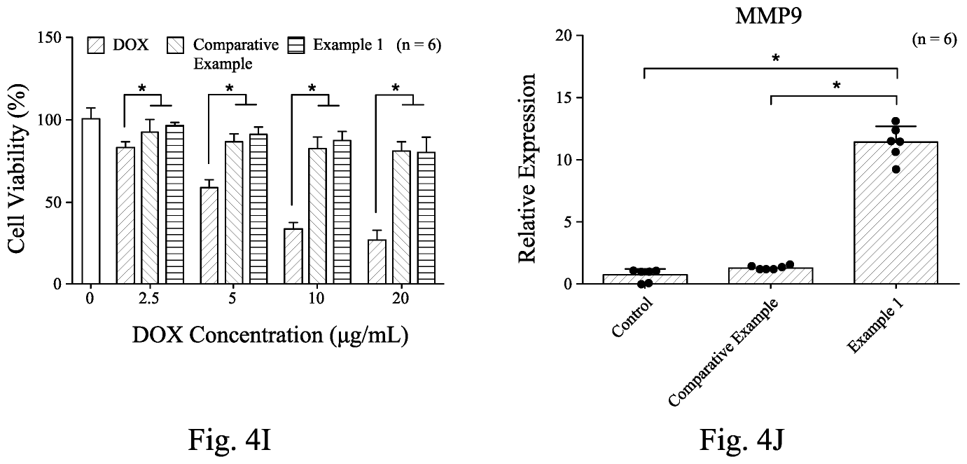

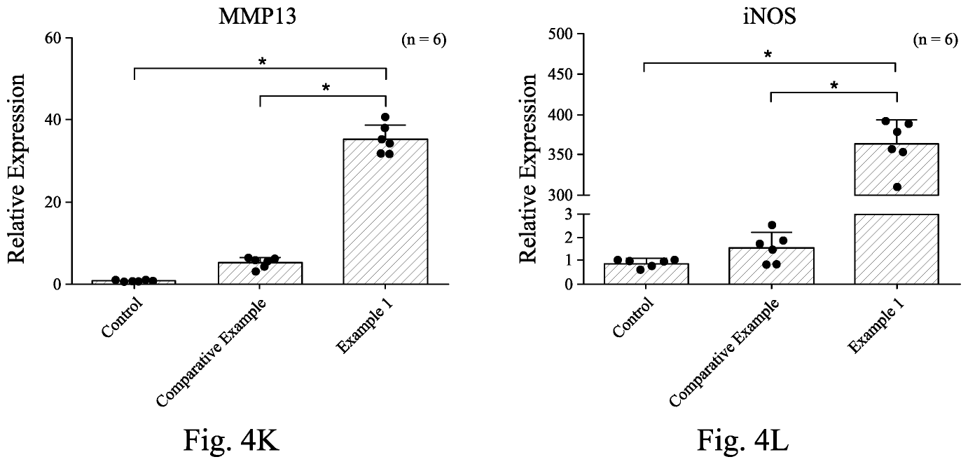

Reference is made to FIG. 4A to FIG. 4D. FIG. 4A, FIG. 4B and FIG. 4C are respectively CLSM images of the RAW264.7 cells treated with free DOX, Comparative Example and Example 1 at different incubation time intervals, and FIG. 4D is a statistical chart showing time-dependent mean fluorescence intensities (MFI) of DOX fluorescence signals of Comparative Example and Example 1 accumulated in the RAW264.7 cells, wherein n.s. represents no significant difference (P>0.05), * represents P<0.05.

In FIG. 4A to FIG. 4D, free DOX, emitting a red fluorescence signal, rapidly diffuse into cells and then accumulated in their nuclei, causing DNA damage, eventually inducing cell apoptosis. In contrast, the cells that had received Example 1 emitted a significantly stronger DOX fluorescence signal, owing to recognition of Dectin-1 on Mφ by the β-glucan in the outer layer of Example 1, than those that have received Comparative Example (P<0.05). The intensity of the fluorescence signal of the intracellular DOX in the RAW264.7 cells treated with Example 1 peaks at 4 hours. The intracellular DOX is predominantly co-localized with the perinuclear lysosomal compartments (green fluorescent signal), suggesting that the most of the internalized particles accumulated in lysosomes. Notably, the accumulation of DOX fluorescence in the cell nuclei is not observed when DOX is delivered by a particulate vehicle of Comparative Example or Example 1.

To verify whether the oral pharmaceutical composition of the present disclosure can be transported by lysosomes in Mφ, the RAW264.7 cells are seeded in an eight-well chamber slide at a density of 5×10⁴ cells per well, incubated overnight, and treated with Example 1 (10 μg/mL DOX). Following incubation for pre-determined periods (0, 2, 4, and 6 hours), the cells are washed with DPBS and stained with LysoTracker™ Red DND-99 (Invitrogen), Tubulin Tracker™ Deep Red (Invitrogen), and Hoechst 33342 for 30 minutes in the incubator. The stained cells are then examined using CLSM to visualize their lysosome trafficking.

Reference is made to FIG. 4E and FIG. 4F. FIG. 4E shows CLSM images of the RAW264.7 cells treated with Example 1 at different incubation time intervals, in which the red fluorescent signal represents the localization of DOX distribution, the yellow fluorescent signal represents the localization of lysosome distribution, the green fluorescent signal represents the localization of microtubule distribution, the blue fluorescent signal represents the localization of the nucleus, and the red lines indicate the computed distances from the center of the lysosome to the pixel that represents the boundary of the nucleus. FIG. 4F is the statistical chart after quantification, where the scale bar represents 10 μm, and * represents P<0.05.

In FIG. 4E and FIG. 4F, the lysosomes that contained Example 1 (yellow fluorescent signal) that have originally been localized to the perinuclear zone begin to translocate toward the cell periphery along the cytoskeletal microtubules (green fluorescent signal). As time pass, more lysosomes farther from the nucleus (blue fluorescent signal) are detected. The translocated lysosomes eventually fuse with the plasma membrane, facilitating efflux of their cargo to the extracellular environment.

To obtain their lysosomal efflux profiles of DOX, the RAW264.7 cells are seeded in a 12-well plate at a density of 2×10⁵ cells per well, incubated overnight, and treated with free DOX, Comparative Example or Example 1 at an equivalent dose of DOX (10 μg/mL). Twenty-four hours later, the medium is replaced with fresh one to remove the test samples. Subsequently, 100 μL of the medium is withdrawn and replaced with fresh medium at pre-determined time points (0, 1, 2, 4, 6, 12, 24, and 48 hours). The intensities of fluorescence (DOX) in the collected media are measured by SpectraMax M5 Microplate Reader. The corresponding lysosomal efflux profiles of Cathepsin D (CTSD) from the treated cells are obtained by a similar method using a Mouse Cathepsin D ELISA Kit (Abcam).

Reference is made to FIG. 4G and FIG. 4H, FIG. 4G shows profiles of lysosomal exocytosis of DOX in the RAW264.7 cells treated with free DOX, Comparative Example and Example 1, and FIG. 4H shows profiles of lysosomal exocytosis of CTSD in the RAW264.7 cells treated with free DOX, Comparative Example and Example 1, wherein * represents P<0.05.

In FIG. 4G, after removing Comparative Example and Example 1 by refreshing the culture medium at 24 hours following their co-incubation with the RAW264.7 cells, the DOX fluorescence in the supernatant increases over time, revealing the lysosomal efflux of DOX. Furthermore, the efflux pattern of DOX is similar to that of CTSD (FIG. 4H), a proteinase that resides inside lysosomes, supporting the finding that the DOX efflux primarily involves lysosomal exocytosis. Notably, the amounts of DOX and CTSD efflux from the RAW264.7 cells treated with Example 1 significantly exceeds those from the RAW264.7 cells treated with Comparative Example (P<0.05). This observation is attributable to the fact that the number of Example 1 that are phagocytosed by the RAW264.7 cells is significantly higher than that of Comparative Example that are phagocytosed.

The capacity of Mφ as a hitchhiking vehicle is strongly correlated with their viability following the uptake of drug-encapsulated particles. Therefore, the cytotoxicity of Example 1 on Mφ are further evaluated. The RAW264.7 cells are incubated with Comparative Example or Example 1 including different DOX concentrations for 24 hours, and the RAW264.7 cells treated with the same concentration of free DOX are used as Control (represents as “DOX”).

Reference is made to FIG. 4I, which shows analysis results of cytotoxicity of free DOX, Comparative Example and Example 1 including different concentrations of DOX to the RAW264.7 cells, wherein * represents P<0.05. In FIG. 4I, the cell viability of the RAW264.7 cells treated with free DOX decreases substantially in a dose-dependent manner, owing to their nuclear DOX accumulation (as in FIG. 4A). Conversely, the RAW264.7 cells treated with Comparative Example or Example 1 retain reasonable cell viability up to a DOX concentration of 20 μg/mL (≥80%), indicating that the pre-loading of Mφ with Comparative Example or Example 1 would not kill them because of their lack of nuclear DOX accumulation.

When activated, Mφ can produce matrix metalloproteinases (MMPs), including MMP9 and MMP13, which regulate extracellular matrix (ECM) degradation. The reverse transcription polymerase chain reaction (RT-PCR) is used to determine the mRNA levels of MMP9 and MMP13 of the RAW264.7 cells that had been treated with Comparative Example or Example 1 for 24 hours.

Reference is made to FIG. 4J and FIG. 4K, FIG. 4J shows analysis result of relative expression of MMP9 in the RAW264.7 cells treated with Comparative Example and Example 1, and FIG. 4K shows analysis result of the relative expression of MMP13 in the RAW264.7 cells treated with Comparative Example and Example 1, wherein * represents P<0.05. In FIG. 4J and FIG. 4K, the RAW264.7 cells treated with Example 1 exhibit markedly higher mRNA levels of MMP9 and MMP13 than the RAW264.7 cells treated with Comparative Example (P<0.05), revealing that the oral pharmaceutical composition of the present disclosure can activate Mφ to form MMPs, so the Mφ that hitchhike Example 1 (hereinafter referred to as “Example 1@Mφ”) can degrade the ECM components in the tumor microenvironment (TME).

Mφ can be polarized into a typical activation M1-like phenotype (hereinafter referred to as “M1Mφ”) or an alternative activation M2-like phenotype (hereinafter referred to as “M2Mφ”). Mφ has plasticity and can be regulated by signal substances secreted by specific cells, tissues or microenvironment in the body, showing continuous phenotype distribution between M1Mφ and M2Mφ. M1Mφ is considered to be a phenotype that promotes inflammation, and clears invading pathogens through phagocytosis and secretion of cytokines that promote inflammation, and is also considered to be a phenotype that suppresses tumors. On the contrary, M2Mφ is considered to be a phenotype that inhibits inflammation and promotes tissue repair. Tumor-associated macrophage (TAM) has been proven to play an important role in various human cancers, which may lead to lymphatic metastasis and distant metastasis, promote tumor growth, develop resistance to chemotherapy drugs, etc., and then worsen the prognosis. TAM is considered to be closer to M2Mφ.

Reference is made to FIG. 4L, which shows analysis results of relative expression of inducible nitric oxide synthase (iNOS) in the RAW264.7 cells treated with Comparative Example and Example 1, iNOS is a biomarker of M1Mφ, and * represents P<0.05. In FIG. 4L, the expression of iNOS in the RAW264.7 cells treated with Example 1 is significantly higher than that in the RAW264.7 cells treated with Comparative Example, indicating that the Mφ activated by Example 1 had been polarized into M1Mφ. NO, which is synthesized by iNOS, is known to be pro-inflammatory and so may be a critical tumoricidal agent. The aforementioned in vitro results reveal that Mφ may function as useful cellular vehicles to hitchhike Example 1 and subsequently be activated by them to produce MMPs and be differentiated toward M1Mφ for in vivo cancer therapy.

### II. Therapeutic Effect of the Oral Pharmaceutical Composition and the Kit for Treating Cancer of the Present Disclosure

### 2.1. Biodistribution of the Oral Pharmaceutical Composition in Experimental Animals

To evaluate the antitumor efficacy of Example 1, an orthotopic pancreatic ductal adenocarcinoma model (hereinafter referred to as “orthotopic PDAC mice”) is first established in the experiment, and then biodistribution of Comparative Example and Example 1 following oral administration of Comparative Example and Example 1 in the orthotopic PDAC mice is observed. Pancreatic cancer can be mainly divided into pancreatic ductal adenocarcinoma and pancreatic neuroendocrine carcinoma. Clinically, 90-95% of patients belong to pancreatic ductal adenocarcinoma. Regardless of the stage of pancreatic cancer patients, five-year survival rate is only about 2-5%.

In the biodistribution study, the orthotopic PDAC mice that had fasted overnight are orally treated with Comparative Example or Example 1 that included 200 μg DOX. The orthotopic PDAC mice that had been intravenous injected with an equivalent dose of free DOX are Control. One hour following intravenous injection of DOX or 6 hours after oral gavage of Comparative Example or Example 1, the fluorescence signals showing distribution and accumulation of DOX in heart, lung, liver, spleen, tumor (PDAC) and kidney of the orthotopic PDAC mice are analyzed by in vivo imaging system (IVIS). The region-of-interest (ROI) is used to quantify the obtained DOX fluorescence signals. To visualize further the time-dependent accumulation of DOX in the PDAC tumor, the orthotopic PDAC mice that had been orally treated with Example 1 are sacrificed at different times (2, 4, 6, and 8 hours) and their pancreases are harvested and examined by IVIS.

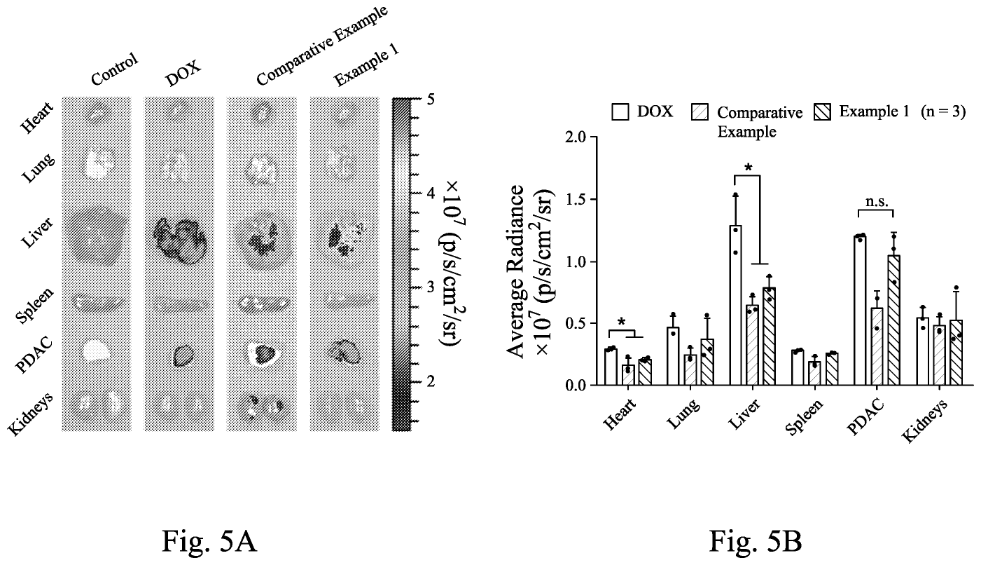

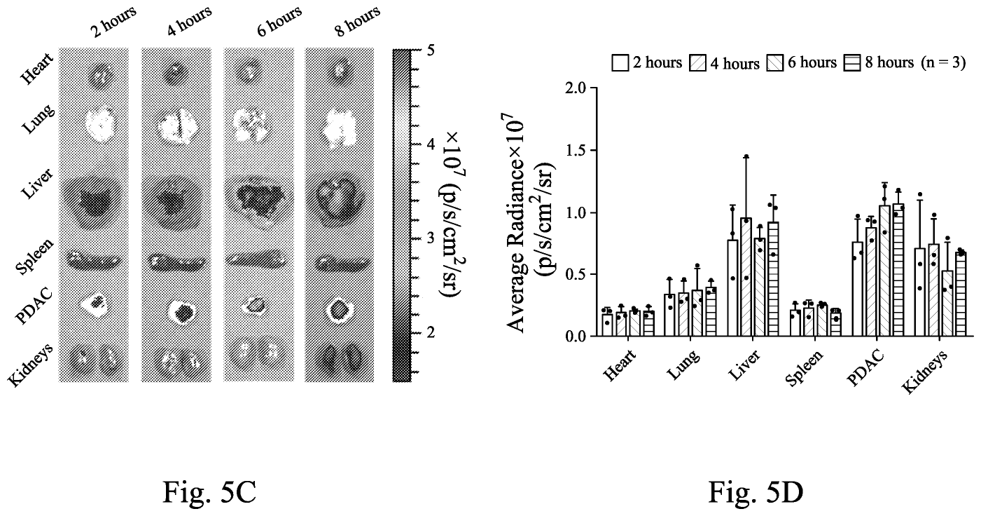

Reference is made to FIG. 5A and FIG. 5B, which shows analysis results of the biodistribution of Comparative Example and Example 1 in the orthotopic PDAC mice. FIG. 5A is an IVIS image, FIG. 5B shows the statistical result of FIG. 5A, n.s. represents no significant difference (P>0.05), and * represents P<0.05.

In FIG. 5A and FIG. 5B, ex vivo IVIS images reveal that the orthotopic PDAC mice that had received Example 1 emitted a substantially stronger DOX fluorescence signal from the PDAC tumor site than those that had received Comparative Example (P<0.05), showing that the oral pharmaceutical composition of the present disclosure exhibits increased tumoral accumulation of the organic compound encapsulated therein. In spite of the effectiveness of DOX in chemotherapy, DOX-induced cardiotoxicity in clinical settings had been frequently reported. Notably, oral treatment with Comparative Example or Example 1 result in significantly less DOX fluorescence in the heart than Control with intravenous treatment with free DOX (P<0.05).

Reference is made to FIG. 5C and FIG. 5D, which show analysis results of biodistribution of Example 1 in the orthotopic PDAC mice at different time points. FIG. 5C is an IVIS image, and FIG. 5D shows the statistical result of FIG. 5C. In FIG. 5C and FIG. 5D, within 2 hours after the orthotopic PDAC mice are oral administered Example 1, DOX fluorescence signals can be observed in the tumor site. The fluorescence intensity at the tumor site increases considerably over time, peaking at 6 hours post-treatment.

The above data support the oral pharmaceutical composition of the present disclosure can target and transpass intestinal M cells, overcome the intestinal epithelial barrier (IEB), and then undergo phagocytosis by endogenous Mφ to form Example 1@Mφ. As hitchhiking cellular vehicles, Example 1@Mφ pass through the ILS and enter systemic circulation, ultimately accumulating in the desmoplastic PDAC tumor tissue because of the tumor-homing and stealth properties that are conferred by Mφ. Accordingly, the Mφ-hitchhiked orally administered the oral pharmaceutical composition of the present disclosure (oral pharmaceutical composition@Mφ) can serve as a precision-guided stealth missile delivery platform for targeted antitumor therapy.

### 2.2. Absorption Mechanisms of the Oral Pharmaceutical Composition

To verify the transport route of Example 1 in vivo, the orthotopic PDAC mice fasted overnight are orally gavaged with Example 1 included 200 μg DOX as w/o inhibitor group, and the orthotopic PDAC mice in w/inhibitor group are treated with 3 mg/kg cycloheximide by intraperitoneal injection, and are orally gavaged 1 hour later with Example 1 including 200 μg DOX. Six hours after oral administrations of Example 1, the orthotopic PDAC mice are sacrificed and Peyer's plaques in the small intestine are collected and immunofluorescent stained. Then the colocalization of Example 1 with M cells and Mφ is detected by CLSM.

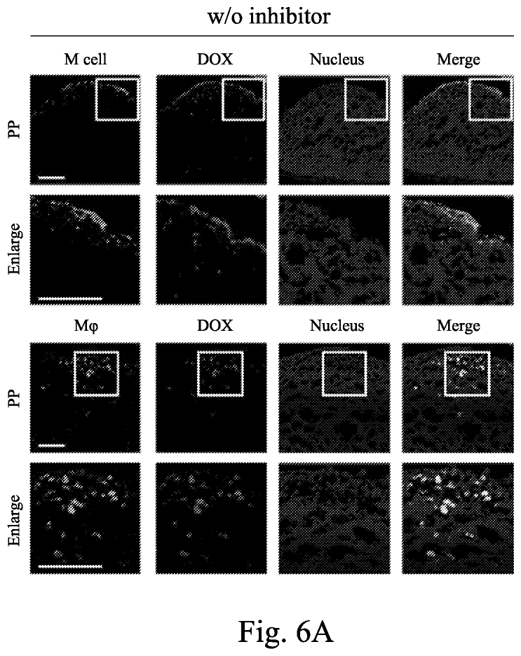

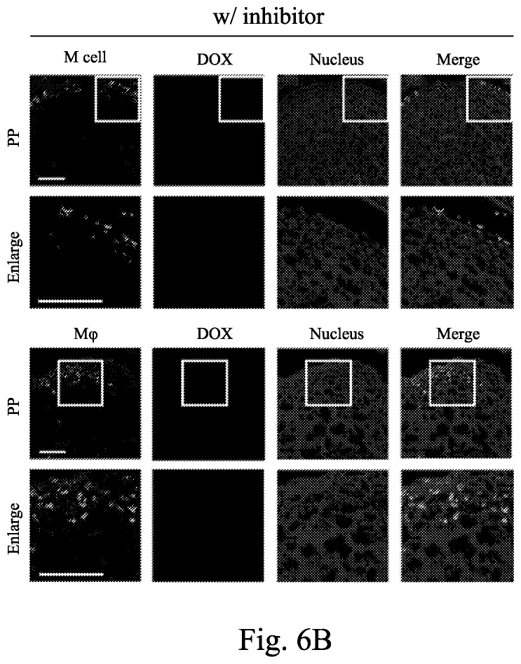

Reference is made to FIG. 6A and FIG. 6B, which show analysis results of the absorption mechanism of Example 1. FIG. 6A shows results of immunofluorescence staining of the w/o inhibitor group, and FIG. 6B shows results of immunofluorescence staining of the w/ inhibitor group. In FIG. 6A, the red fluorescence signal of DOX and the localization of the green fluorescence of marked M cells in the stained cells show that Example 1 can target and transpass the intestinal M cells. In addition, the red fluorescent signal of DOX and the green fluorescent signal of the marked Mφ also have a clear intracellular colocalization, showing that Example 1 can be phagocytosed by Mφ and enter the lymphatic system. However, the aforementioned phenomenon is not observed in the w/inhibitor group due to the addition of inhibitor.

### 2.3. Use of the Oral Pharmaceutical Composition for Treating Early Stage Pancreatic Cancer

### 2.3.1. Antitumor Efficacy of the Oral Pharmaceutical Composition

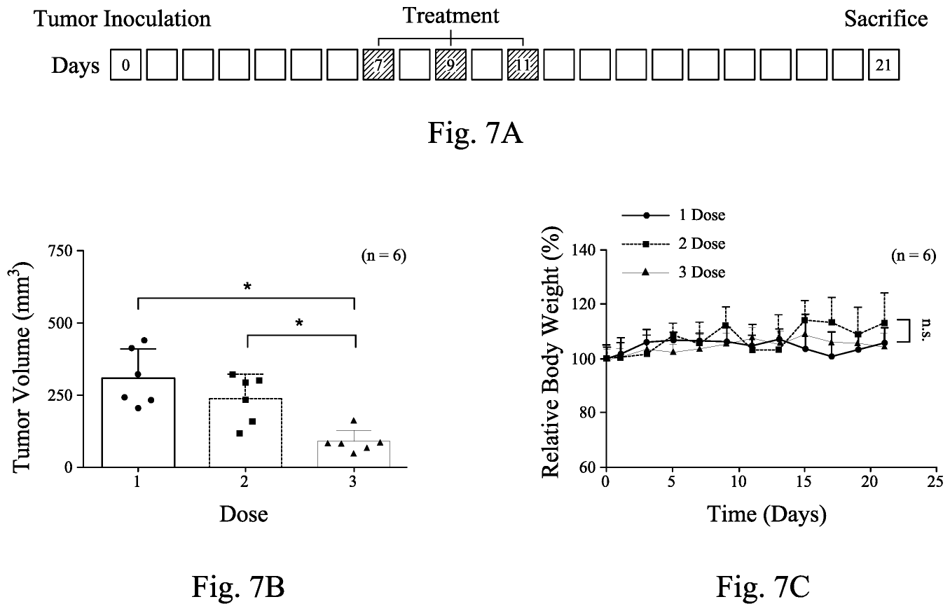

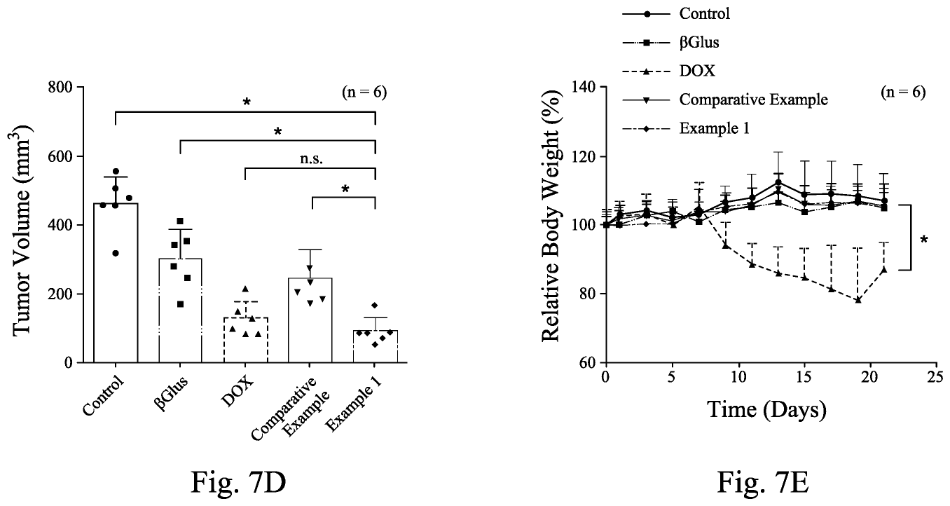

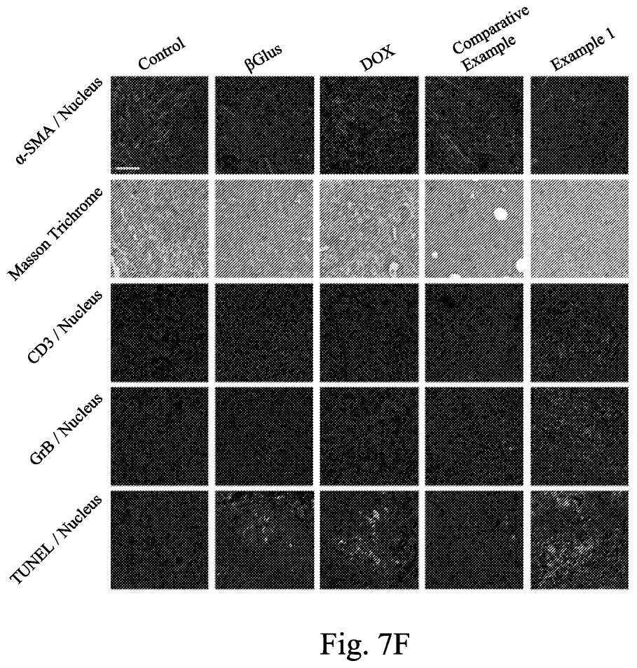

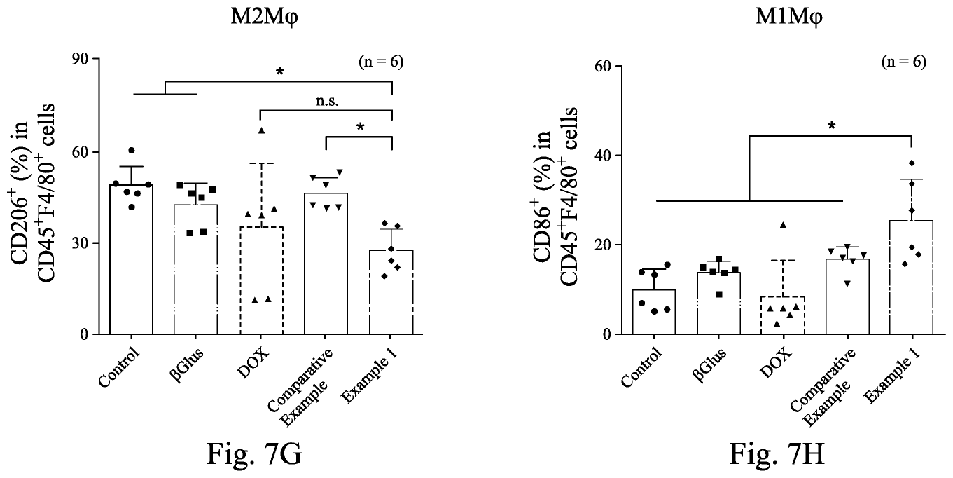

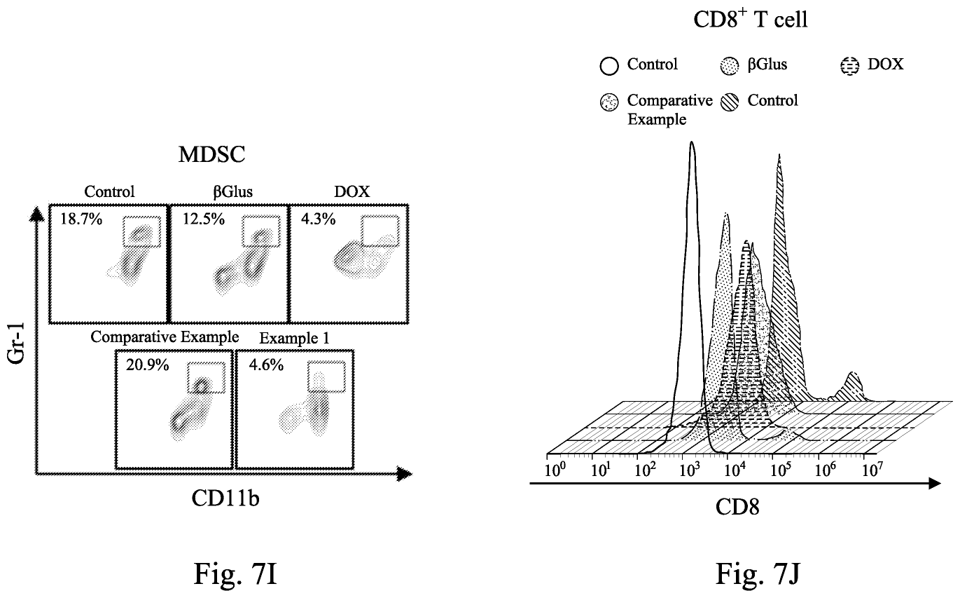

Before the antitumor efficacy of Example 1 is assessed, the dose-dependent effect of Example 1 on tumors was evaluated in orthotopic PDAC mice. Reference is made to FIG. 7A, which is a schematic view showing dose-dependent therapeutic strategies for tumors. The orthotopic PDAC mice are orally treated with Example 1 using one dose on day 7, each of days 7 and 9 (two doses), or each of days 7, 9 and 11 (three doses), following inoculation of the tumors. On day 21, the orthotopic PDAC mice are euthanized and their PDAC tumors are retrieved for analysis. The volume of each retrieved tumor is calculated as length×width×height×π/6; all dimensions were measured using a pair of calipers.

Reference is made to FIG. 7B and FIG. 7C, which show the tumor volume and body weight curves of the orthotopic PDAC mice treated with different doses of Example 1, wherein n.s. represents no significant difference (P>0.05), and * represents P<0.05. At the experimental endpoint on day 21, a dose-dependent reduction of the tumor volume is detected in the mice, revealing the therapeutic effectiveness of the orally administered Example 1. The orthotopic PDAC mice that had received three doses exhibit the greatest reduction in tumor volume (P<0.05) and their body weights are comparable to those of the orthotopic PDAC mice that had received one or two doses (P>0.05). Thus, a three-dose treatment schedule is used in subsequent experiments.

To evaluate the antitumor efficacy of different treatments, the orthotopic PDAC mice are randomly assigned to the following groups: untreated control group (hereinafter referred to as “Control”), orally administered β-glucan group (hereinafter referred to as “βGlus”), intravenous injected DOX group (hereinafter referred to as “DOX”), orally administered Comparative Example group (hereinafter referred to as “Comparative Example”), and orally administered Example 1 group (hereinafter referred to as “Example 1”). In the groups of DOX, Comparative Example and Example 1, the DOX content of each dose is 10 mg/kg, while in the groups of βGlus and Example 1, the content of β-glucan of each dose is 40 mg/kg. The body weight changes of the orthotopic PDAC mice are recorded every other day. On day 21, the orthotopic PDAC mice are euthanized and their tumors are retrieved for analysis.

Reference is made to FIG. 7D and FIG. 7E, which show the tumor volume and body weight curves of the orthotopic PDAC mice with different treatments, wherein n.s. represents no significant difference (P>0.05), and * represents P<0.05. In FIG. 7D and FIG. 7E, although the volume of the tumor is reduced in βGlus or Comparative Example, the treatment effect is not statistically significant compared with Control. βGlus have been shown to promote pro-inflammatory responses, converting Mφ into an M1Mφ, and have therefore been used as an adjuvant in cancer therapy. In contrast, DOX or Example 1 substantially reduces tumor volumes (P<0.05). Although DOX has significant antitumor effectiveness, the result in FIG. 7E shows that the orthotopic PDAC mice that are intravenous injected DOX suffered serious loss of body weight (P<0.05), suggesting the toxicity caused by the treatment with intravenous DOX can have a great impact on experimental animals. Notably, Example 1-treated mice exhibit greater reductions of tumor volumes than Comparative Example-treated mice (P<0.05). This finding reveals that Example 1@Mφ is functioned as cellular tumor-seeking stealth missiles, has significantly higher antitumor efficacy than the oral treatment of Comparative Example.

### 2.3.2. Tumor Microenvironment Regulation Effect of the Oral Pharmaceutical Composition

Pancreatic ductal adenocarcinoma (PDAC) is one of the deadliest cancers as it metastasizes rapidly and is commonly diagnosed too late. While systemic chemotherapy is the standard treatment for PDAC, the prognosis of this approach is typically poor because the tumor microenvironment (TME) is highly desmoplastic. Studies have revealed that PDAC tumor cells can elicit a desmoplastic response at both the primary and metastatic lesions by stimulating cancer-associated fibroblasts (CAFs) to elevate the excessive expression of extracellular matrix (ECM) components, such as collagen, around tumor tissues. A dense stromal barrier is thus formed, preventing penetration of the drug into the tumor tissue, reducing therapeutic efficacy. PDAC features a substantial elevation of the proliferation/activation of α-smooth muscle actin (α-SMA)-positive CAFs and an increased deposition of many ECM components, including collagen, in the TME.

To assess the effects of each treatments modality on the modulation of the PDAC-specific stromal TME, the tumor tissues of the orthotopic PDAC mice are harvested following various treatments and processed for immunohistochemical staining against CAFs with α-SMA antibody. Masson's trichrome staining is conducted to evaluate the extent of collagenous fibers around tumor tissues. CD3 antibody and granzyme B (GrB) are used for immunohistochemical staining to evaluate CD3⁺ T cell infiltration and granzyme B secretion, and TUNEL staining is performed to observe the apoptosis of tumor cells.

Reference is made to FIG. 7F, which shows histological analysis results of tumors of the orthotopic PDAC mice administered with different treatments, wherein the scale bar represents 100 μm. In FIG. 7F, less α-SMA expression, suggesting improved suppression of the proliferation/activation of CAFs, and a greater reduction of collagen content, indicating enhanced stromal depletion, are detected in the orthotopic PDAC mice treated with Example 1 than in the orthotopic PDAC mice treated with Control. These findings likely follow from the fact the oral pharmaceutical composition of the present disclosure can activate their hitchhiking cellular vehicles (Mφ), causing a shift of their MMP profile to deplete actively fibrosis in the tumor stroma. In response to stromal TME remodeling, the group that had been orally treated with Example 1 exhibits more CD3⁺ T cell infiltration and granzyme B secretion than Control, so it has more apoptotic tumor cells, as revealed by the TUNEL assay.

The TME in a grown tumor is typically immunosuppressive. To determine whether the TME of the orthotopic PDAC mice that had received Example 1 could be modified from immunosuppressive to immunogenic, the populations of regulatory cells, including M1Mφ, M2Mφ and myeloid-derived suppressor cells (MDSCs), that inhibit the activation of effector T cells in tumor tissues, are analyzed by flow cytometry.

Reference is made to FIG. 7G to FIG. 7J, FIG. 7G is a statistical chart of the percentage of M2Mφ in different treatment groups, FIG. 7H is a statistical chart of the percentage of M1Mφ in different treatment groups, FIG. 7I shows representative flow histogram plots of MDSC in different treatment groups, and FIG. 7J shows representative flow histogram plots of CD8⁺ T cells in different treatment groups, wherein n.s. represents no significant difference (P>0.05), and * represents P<0.05.

In FIG. 7G to FIG. 7J, in comparison with Control, the group that had orally received Example 1 has significantly fewer M2Mφ and MDSCs but more M1Mφ and greater infiltration of CD8⁺ T cells in the tumor tissue, indicating that administration of Example 1 can convert the immunosuppressive TME of the orthotopic PDAC mice to immunogenic. The above results reveal that the “precision-guided stealth missile”-like Example 1@Mφ effectively reduced the tumor stroma fibrosis and modulated the TME, reducing the levels of regulatory cells, enhancing the recruitment of effector T cells, and eventually inducing the apoptosis of tumor cells, leading to improved antitumor efficacy.

### 2.4. Use of the Kit for Treating Cancer for Treating Early Stage Pancreatic Cancer

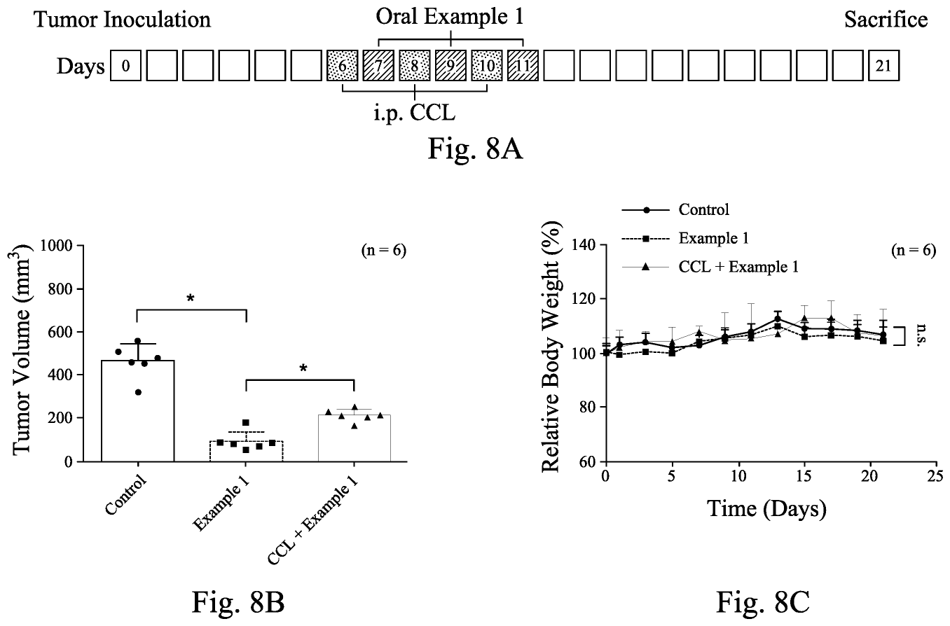

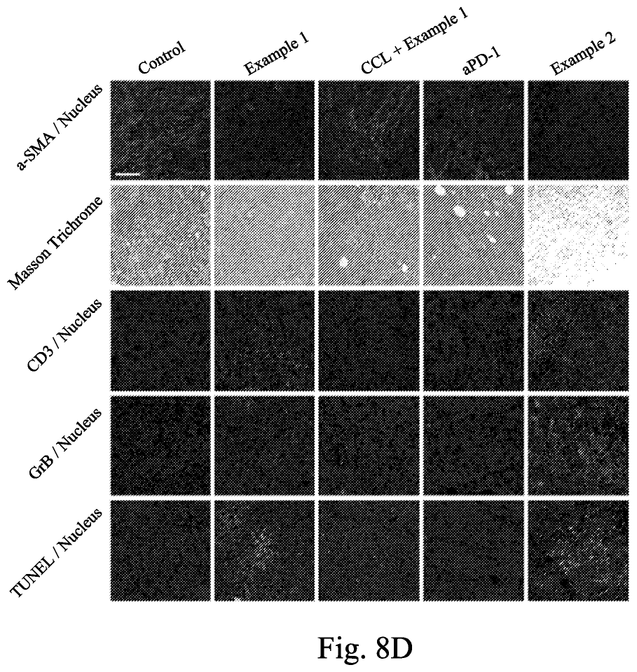

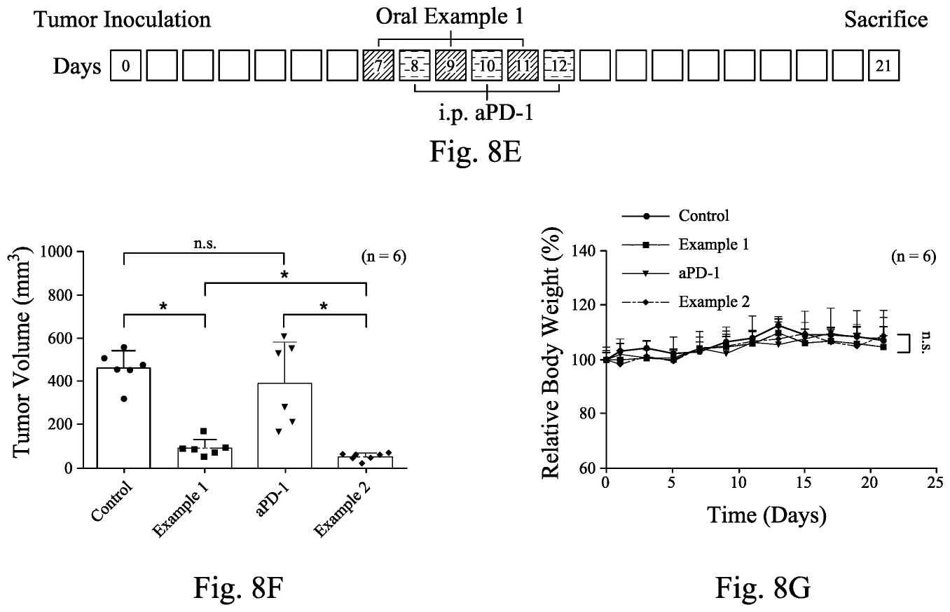

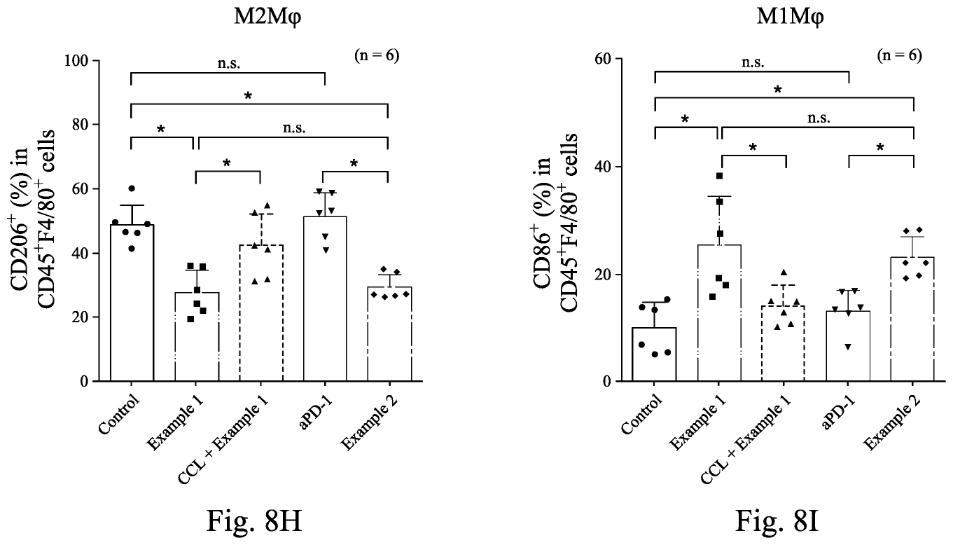

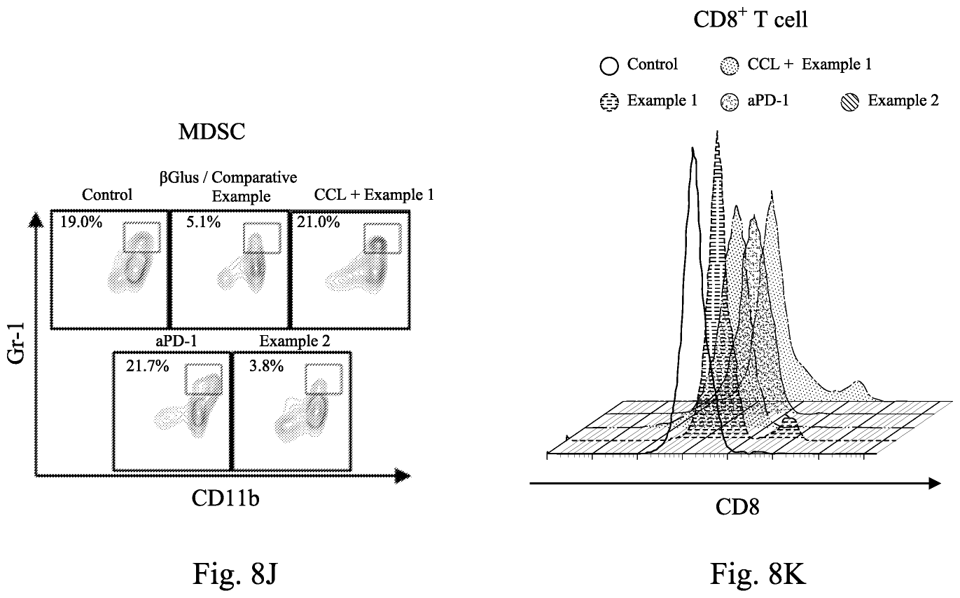

To verify the importance of endogenous Mφ in the Example 1-mediated antitumor effect, the orthotopic PDAC mice are pre-treated via intraperitoneal injection with clodronate-containing liposomes (CCL), which have been used elsewhere to deplete Mφ systemically, before they are orally treated with Example 1. Reference is made to FIG. 8A, which is a schematic view of the treatment strategy of CCL and/or Example 1 to treat the orthotopic PDAC mice. After the mice were inoculated with tumors, the groups of Example 1 and CCL+Example 1 are orally treated with Example 1 using three doses on each of days 7, 9 and 11. In the group of CCL+Example 1, the orthotopic PDAC mice are intraperitoneal injected with 75 mg/kg CCL on each of days 6, 8 and 10. On day 21, all groups of the orthotopic PDAC mice are euthanized and their PDAC tumors are retrieved for analysis. The volume of each retrieved tumor is calculated as length ×width×height×π/6; all dimensions were measured using a pair of calipers.

Reference is made to FIG. 8B to FIG. 8D. FIG. 8B and FIG. 8C show the tumor volume and body weight curves of the orthotopic PDAC mice with different treatments, and FIG. 8D shows histological analysis results of tumors of the orthotopic PDAC mice administered with different treatments, wherein n.s. represents no significant difference (P>0.05), * represents P<0.05, and the scale bar represents 100 μm. In FIG. 8B and FIG. 8C, no apparent loss of body weight is observed in any of the treated groups. Larger tumors are detected in the orthotopic PDAC mice that had been treated with CCL+Example 1 (P<0.05) than in the orthotopic PDAC mice that had been treated with Example 1 alone. Additionally, in FIG. 8D, following the depletion of Mφ, the reduction of stroma fibrosis, accumulation of T cells, and induction of cell apoptosis in tumor tissues that are induced by oral treatment with Example 1 are impaired. Collectively, these results show that the TME modulation and antitumor effect of the oral pharmaceutical composition of the present disclosure are Mφ-dependent.

In spite of success of immune checkpoint blockade (ICB) in treating various cancers, the therapeutic efficacy of ICB therapies in treating PDAC is limited, owing to highly desmoplastic TME of PDAC. The effectiveness of the kit for treating cancer (including the oral pharmaceutical composition of the present disclosure and the immune checkpoint blockade) in treating desmoplastic PDAC.

The oral pharmaceutical composition of the present disclosure used in the experiment is Example 1, and the immune checkpoint blockade used is PD-1 antibody (hereinafter referred to as “aPD-1”). Reference is made to FIG. 8E, which is a schematic view of a therapeutic strategy for the orthotopic PDAC mice treated with the oral pharmaceutical composition of the present disclosure and the kit for treating cancer of the present disclosure. After the mice were inoculated with tumors, the groups of Example 1 and aPD-1+Example 1 (hereinafter referred to as “Example 2”) are orally treated with Example 1 using three doses on each of days 7, 9 and 11. In the group of aPD-1 and Example 2, the orthotopic PDAC mice are intraperitoneal injected with 10 mg/kg aPD-1 on each of days 8, 10 and 12. On day 21, all groups of the orthotopic PDAC mice are euthanized and their PDAC tumors are retrieved for analysis. The volume of each retrieved tumor is calculated as length×width×height×π/6; all dimensions were measured using a pair of calipers.

Reference is made to FIG. 8D and FIG. 8F to FIG. 8K. FIG. 8D shows histological analysis results of tumors of the orthotopic PDAC mice administered with different treatments; FIG. 8F and FIG. 8G show the tumor volume and body weight curves of the orthotopic PDAC mice with different treatments; FIG. 8H is a statistical chart of the percentage of M2Mφ in different treatment groups; FIG. 8I is a statistical chart of the percentage of M1Mφ in different treatment groups; FIG. 8J shows representative flow histogram plots of MDSC in different treatment groups, and FIG. 8K shows representative flow histogram plots of CD8⁺ T cells in different treatment groups, wherein n.s. represents no significant difference (P>0.05), and * represents P<0.05.

In FIG. 8F and FIG. 8G, no apparent loss of body weight is observed in any of the treated groups. No significant difference in tumor volume is observed between the aPD-1-treated group and the Control (P>0.05), suggesting that PD-1 blockade alone by treating aPD-1 is unsatisfactory in treating PDAC. In contrast, Example 2 exhibits a remarkable reduction in tumor volume below those in the control groups (Control, Example 1, and aPD-1, P<0.05), indicating a markedly improved therapeutic response to PD-1 blockade. This finding is attributable to the antifibrotic activity of orally treated Example 1, which significantly increases T cell infiltration in the TME, ultimately increasing the antitumor activity of aPD-1. Moreover, in FIG. 8H to FIG. 8K, treatment of Example 2 clearly modifies the immunosuppressive TME, as revealed by the decreased percentages of M2Mφ and MDSCs, the increased percentages of M1Mφ, and the enhanced recruitment of CD8⁺ T cells in the tumor tissue, which induced significant apoptosis in tumor cells.

### 2.5. Use of the Oral Pharmaceutical Composition and the Kit for Treating Cancer for Treating Advanced Pancreatic Cancer

To evaluate the treatment effect of the oral pharmaceutical composition and the kit for treating cancer of the present disclosure on advanced PDAC metastasis, 2×10⁵ cancer cells (murine AK4.4 cell line) are inoculated into the pancreas of each recipient to establish a PDAC model with spontaneous metastasis (hereinafter referred to as “metastatic PDAC mice”). By day 10, the metastatic PDAC mice had developed an orthotopic primary tumor along with metastatic tumor lesions in the lungs and liver. Six hours after the metastatic PDAC mice are orally gavaged with Example 1, the biodistribution and accumulation of DOX fluorescent signals in the heart, lung, liver, spleen, tumor (PDAC) and kidney of the metastatic PDAC mice are confirmed using IVIS. In the experiment, the metastatic PDAC mice without drug administration are also included as Control.

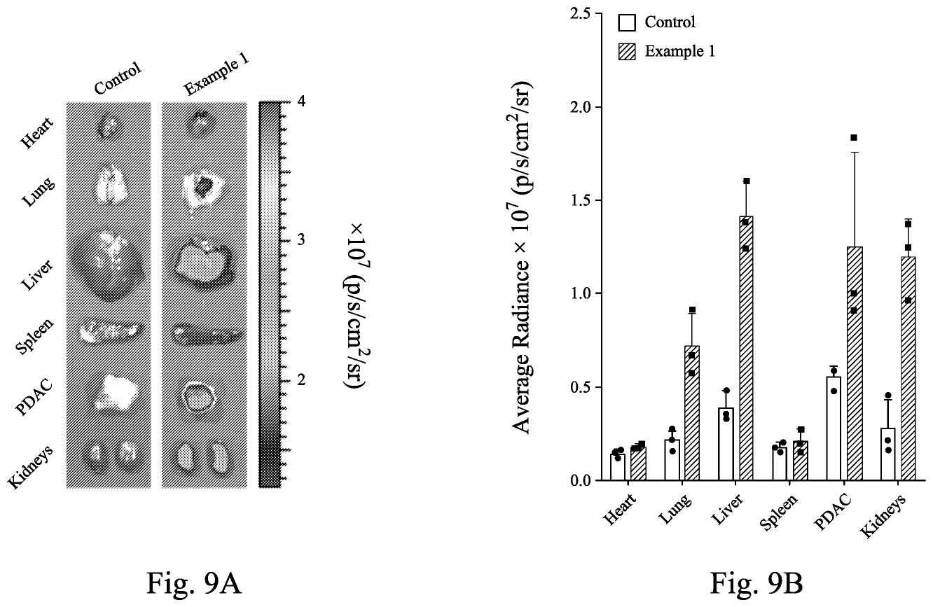

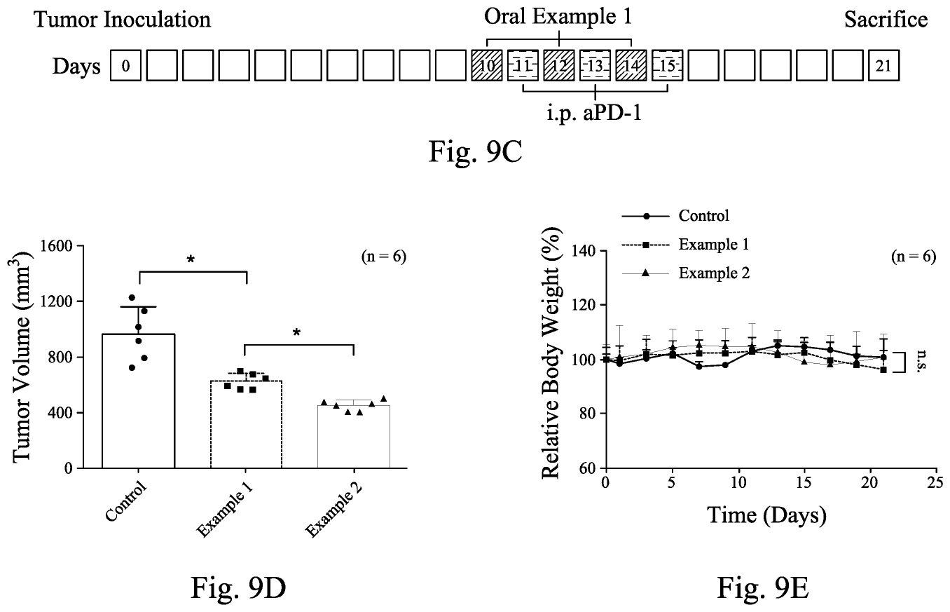

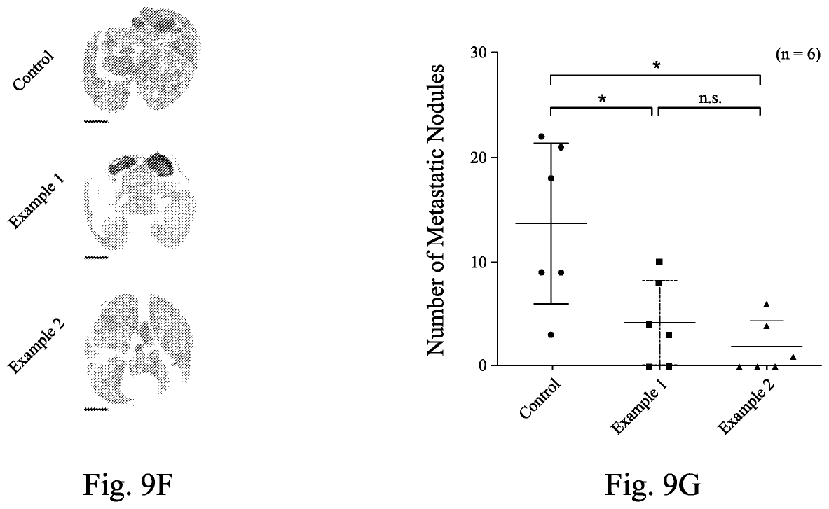

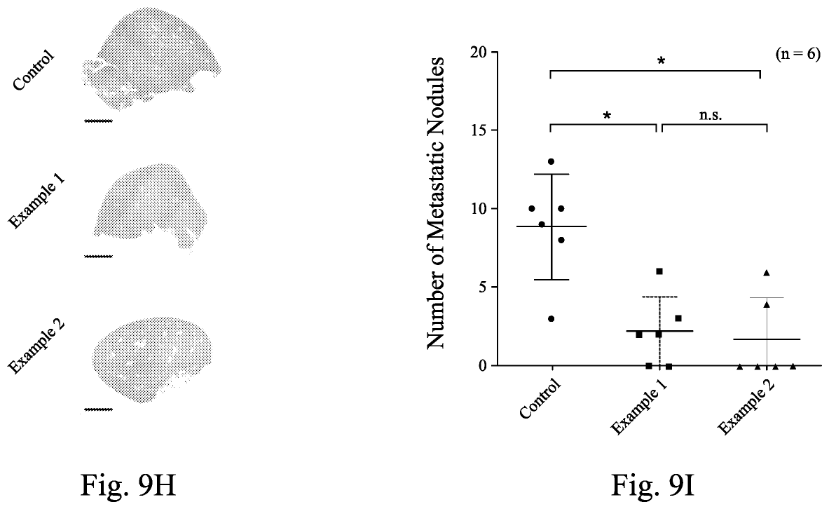

Reference is made to FIG. 9A and FIG. 9B, which shows analysis results of the biodistribution of Control and Example 1 in the metastatic PDAC mice. FIG. 9A is an IVIS image, and FIG. 9B shows the statistical result of FIG. 9A. The result reveals that the metastatic PDAC mice treated with Example 1 have high DOX fluorescence signals in the lung, liver, PDAC, and kidney, showing that orally administered Example 1 specifically target and accumulate in the primary and metastatic tumors, owing to the homing capacity of their hitchhiking cellular vehicles, Mφ which acts as “precision-guided stealth missiles” in targeted antitumor therapy.

Reference is made to FIG. 9C, which is a schematic view of a therapeutic strategy for treating metastatic PDAC mice with the oral pharmaceutical composition of the present disclosure and the kit for treating cancer of the present disclosure. After the mice were inoculated with tumors, the groups of Example 1 and Example 2 are orally treated with Example 1 using three doses on each of days 10, 12 and 14. In the group of Example 2, the metastatic PDAC mice are intraperitoneal injected with 10 mg/kg aPD-1 on each of days 11 13 and 15. On day 21, all groups of the metastatic PDAC mice are euthanized and their PDAC tumors are retrieved for analysis. The volume of each retrieved tumor is calculated as length×width×height×π/6; all dimensions were measured using a pair of calipers. Tumor tissues are then stained with hematoxylin and eosin (H&E) to detect the number of metastatic nodules.

Reference is made to FIG. 9D to FIG. 9I. FIG. 9D and FIG. 9E show the tumor volume and body weight curves of the metastatic PDAC mice with different treatments. FIG. 9F and FIG. 9G show results of H&E staining of the lung of the metastatic PDAC mice with different treatments, wherein FIG. 9F shows H&E staining images, and FIG. 9G is a statistical chart of the number of metastatic nodules. FIG. 9H and FIG. 9I show results of H&E staining of the liver of the metastatic PDAC mice with different treatments, wherein FIG. 9H shows H&E staining images, and FIG. 9I is a statistical chart of the number of metastatic nodules. In FIG. 9D to FIG. 9I, n.s. represents no significant difference (P>0.05), and * represents P<0.05.

The results no apparent loss of body weight is observed in any of the treated groups. Compared with Control, the administration of Example 1 can significantly inhibit the growth of primary tumors in the metastatic PDAC mice (P<0.05), while the administration of Example 2 can more significantly inhibit the growth of primary PDAC (compared with the group treated with Example 1, P<0.05). In FIG. 9F to FIG. 9I, there are many metastatic nodules in the lungs and livers of Control, while the number of metastatic nodules in the lungs and livers of the metastatic PDAC mice treated with Example 1 or Example 2 is significantly reduced (P<0.05). Notably, in the group treated with Example 1, two out of six metastatic PDAC mice did not exhibit metastatic lesions. Whereas, in the group treated with Example 2, four out of six metastatic PDAC mice did not have metastatic lesions. These analytical results reveal that Example 2 (the combined treatment of Example 1@ Mφ and aPD-1) effectively inhibits the growth of primary PDAC and suppressed the development of metastasis.

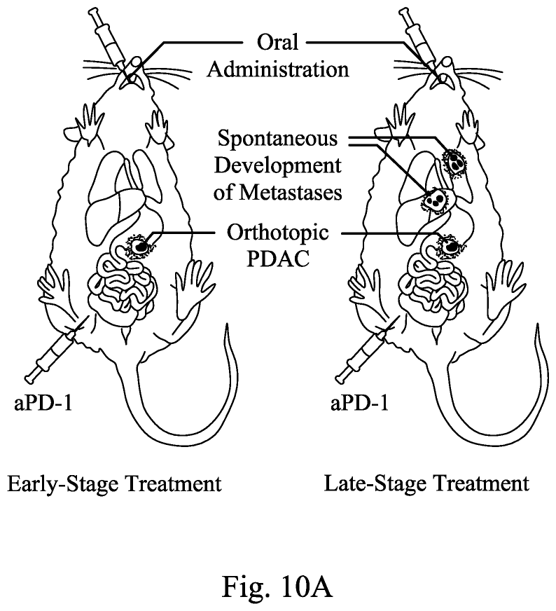

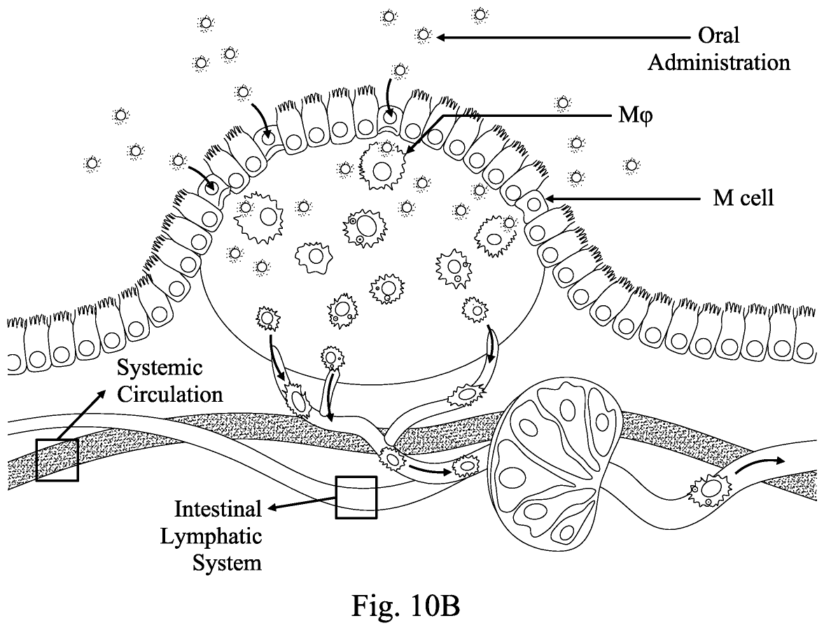

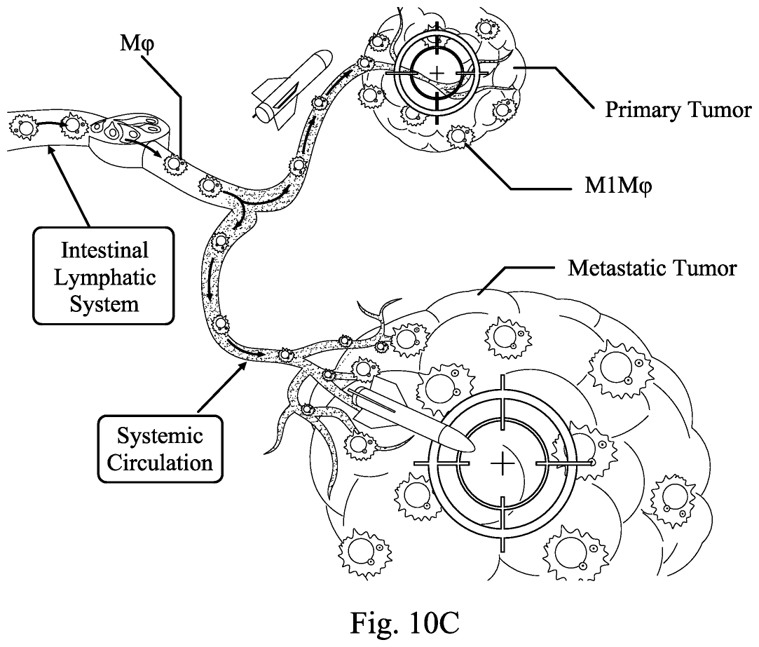

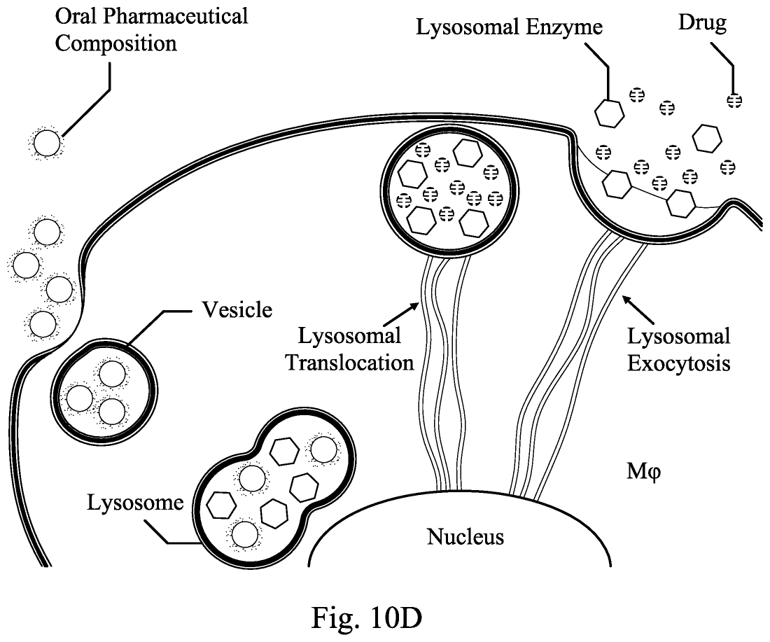

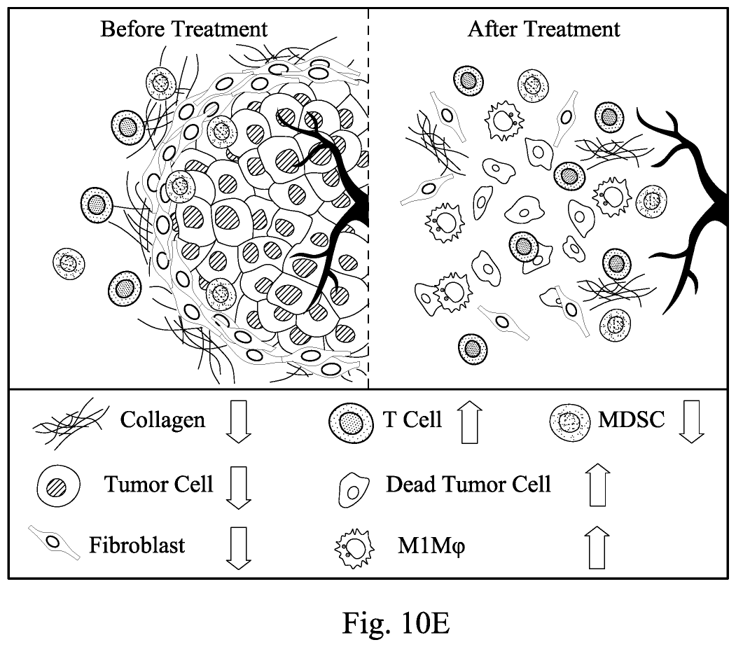

Reference is made to FIG. 10A to FIG. 10E. FIG. 10A is a schematic view showing the oral pharmaceutical composition and the kit for treating cancer of the present disclosure used in the treatment of PDAC model. FIG. 10B is a schematic view showing a mechanism of intestinal absorption of the oral pharmaceutical composition of the present disclosure mediated by M cells. FIG. 10C is a schematic view showing a mechanism of the oral pharmaceutical composition and the kit for treating cancer of the present disclosure targeting primary tumor and metastatic tumor. FIG. 10D is a schematic view showing a mechanism of lysosomal translocation and lysosomal exocytosis of the oral pharmaceutical composition of the present disclosure. FIG. 10E is a schematic view showing an action mechanism of the oral pharmaceutical composition and the kit for treating cancer of the present disclosure remodeling the tumor microenvironment.

The oral pharmaceutical composition of the present disclosure can remain stable in extreme gastrointestinal environment, so the oral pharmaceutical composition and the kit for treating cancer of the present disclosure can be administered orally. When the oral pharmaceutical composition of the present disclosure reaches the intestinal tract, the β-glucan exposed on outer layer of the oral pharmaceutical composition can target the Dectin-1 receptor on the intestinal M cells, and then be absorbed into the body through transcytosis of M cells into the intestinal lymphatic system. The immune cells in the lymphatic system (such as Mφ) also express abundant Dectin-1 on their cellular membranes, which can specifically recognize the β-glucan on the outer layer of the oral pharmaceutical composition of the present disclosure, so that Mφ can phagocytose and hitchhike the oral pharmaceutical composition of the present disclosure. Mφ that hitchhike the oral pharmaceutical composition of the present disclosure responds to tumor-related chemokine/cytokine cues transiting through lymphatic vessels, entering system circulation, eventually homing in on the tumor site. In addition, the oral pharmaceutical composition of the present disclosure can release the encapsulated organic compound via translocation and exocytosis in the lysosome. The DOX encapsulated in Example 1 of the present disclosure generates active oxides in Mφ, thereby promoting Mφ covert to M1Mφ, reversing immunosuppressive tumor microenvironment therein, and finally achieving a combined chemical and immune therapy. Furthermore, when distant metastasis occurs in vivo, Mφ also responds to cytokines generated in each metastatic lesion to achieve same treatment effect as that of orthotopic tumors. In addition, the oral pharmaceutical composition of the present disclosure can combine with an immune checkpoint inhibitor (that is, the kit for treating cancer of the present disclosure) to achieve better anti-tumor effects and inhibit the development of tumor metastasis.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An oral pharmaceutical composition, comprising:
a metal complex formed by coordinate bonding of an organic compound to a metal ion, wherein the organic compound is an aromatic compound and comprises at least two oxygen atoms; and
an outer layer comprising a β-glucan, wherein the β-glucan is dispersed outside the metal complex and coordinately bonded to the metal complex to form the outer layer.

2. The oral pharmaceutical composition of claim 1, wherein the oral pharmaceutical composition is a sphere, and the average particle size of the sphere is greater than or equal to 80 nm and less than 1 μm.

3. The oral pharmaceutical composition of claim 1, wherein the metal ion is zinc ion (Zn²⁺), manganese ion (Mn²⁺), cobalt ion (Co²⁺), iron ion (Fe³⁺) or nickel ion (Ni²⁺).

4. The oral pharmaceutical composition of claim 1, wherein the organic compound is doxorubicin (DOX), retinoic acid, curcumin, quercetin or sorafenib.

5. A method for fabricating an oral pharmaceutical composition, comprising:
providing a mixed solution, wherein the mixed solution comprises an organic compound, a metal ion and a B-glucan, and the organic compound is an aromatic compound and comprises at least two oxygen atoms; and
performing a synthesis step, wherein energy is provided to the mixed solution, then the organic compound coordinates with the metal ion to form a metal complex, and the β-glucan coordinates with the metal complex to form the oral pharmaceutical composition.

6. The method for fabricating the oral pharmaceutical composition of claim 5, wherein a molar ratio of the organic compound and the metal ion in the mixed solution is 0.5:1 to 8:1.

7. The method for fabricating the oral pharmaceutical composition of claim 5, wherein the organic compound is doxorubicin (DOX), retinoic acid, curcumin, quercetin or sorafenib.

8. The method for fabricating the oral pharmaceutical composition of claim 5, wherein the metal ion is formed by a dissociation of a metal salt in water, and the metal ion is zinc ion (Zn²⁺), manganese ion (Mn²⁺), cobalt ion (Co²⁺), iron ion (Fe³⁺) or nickel ion (Ni²⁺).

9. The method for fabricating the oral pharmaceutical composition of claim 5, wherein in the synthesis step, energy is provided by a microwave or a heating.

10. A kit for treating cancer, comprising:
the oral pharmaceutical composition of claim 4; and
a pharmaceutically acceptable carrier.

11. The kit for treating cancer of claim 10, further comprising an immune checkpoint inhibitor.

12. The kit for treating cancer of claim 11, wherein the immune checkpoint inhibitor is selected from the group consisting of PD-L1 antibody, PD-1 antibody, CTLA-4 antibody and TIM-3 antibody.
